(19) United States
(12) Patent Application Publication
(10) Pub. No.: US 2022/0048937 A1
(43) Pub. Date: Feb. 17, 2022

(54) COMPOSITION AND ORGANIC LIGHT-EMITTING DEVICE

(71) Applicant: Sumitomo Chemical Co., Ltd., Tokyo (JP)

(72) Inventors: Martin Humphries, Godmanchester (GB); Kiran Kamtekar, Godmanchester (GB); William Tarran, Godmanchester (GB); Florence Bourcet, Cambridge (GB); James Lee, Godmanchester (GB)

(73) Assignee: Sumitomo Chemical Co., Ltd., Tokyo (JP)

(21) Appl. No.: 17/413,598

(22) PCT Filed: Dec. 12, 2019
(86) PCT No.: PCT/GB2019/053521

§ 371 Date: Jun. 14, 2021

(30) Foreign Application Priority Data
Dec. 14, 2018 (GB) 1820386.9

(51) Int. Cl. C07F 15/00; H01L 51/00; C07D 493/00; C07D 495/00

(52) U.S. Cl. CPC C07F 15/0033; H01L 51/0085; H01L 51/0074; H01L 51/5016; H01L 51/0052; C07D 493/00; C07D 495/00; H01L 51/0073

(57) ABSTRACT

A composition comprising a semiconducting host compound having a glass transition temperature (Tg) of less than 100° C., and a phosphorescent compound wherein the phosphorescent compound is a metal complex of formula (II): M is Ir (III) or Pt (II). p is at least 1. q is 0, 1 or 2. L is a bidentate ligand substituted with one or two groups X wherein each X independently comprises an aromatic or heteroaromatic group Ar⁵; the sum of the number of rings comprised in the one or more X groups of formula (II) is at least 12; and at least 75% of the mass of each X is made up of the mass of the aromatic or heteroaromatic ring atoms of Ar⁵. L² is a bidentate ligand which is different from L¹.

### BACKGROUND

The present disclosure relates to phosphorescent compositions, and organic light-emitting devices containing said compositions.

Electronic devices containing active organic materials are known for use in devices such as organic light emitting diodes (OLEDs), organic photoresponsive devices, organic transistors and memory array devices. Devices containing active organic materials offer benefits such as low weight, low power consumption and flexibility. Moreover, use of soluble organic materials allows use of solution processing in device manufacture, for example inkjet printing or spin-coating.

An OLED may comprise a substrate carrying an anode, a cathode and one or more organic light-emitting layers between the anode and cathode.

Holes are injected into the device through the anode and electrons are injected through the cathode during operation of the device. Holes in the highest occupied molecular orbital (HOMO) and electrons in the lowest unoccupied molecular orbital (LUMO) of a light-emitting material combine to form an exciton that releases its energy as light.

Light-emitting materials include small molecule, polymeric and dendrimeric materials. Light-emitting polymers include poly(arylene vinylenes) such as poly(p-phenylene vinylenes) and polymers containing arylene repeat units, such as fluorene repeat units.

A light emitting layer may comprise a host material and a light-emitting dopant wherein energy is transferred from the host material to the light-emitting dopant. For example, J. Appl. Phys. 65, 3610, 1989 discloses a host material doped with a fluorescent light-emitting dopant (that is, a light-emitting material in which light is emitted via decay of a singlet exciton).

Phosphorescent dopants are also known (that is, a light-emitting dopant in which light is emitted via decay of a triplet exciton).

WO 2017/144863 discloses compounds of formula (III):

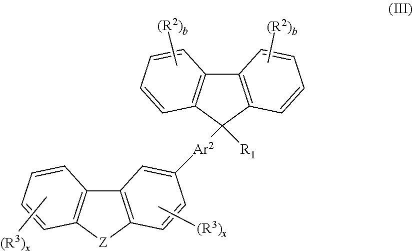

wherein Ar² is an arylene or heteroarylene group; Z is O or S; R¹ is a substituent bound directly to the fluorene unit by an sp³ hybridised carbon atom; R² and R³ are substituents; x is 0, 1, 2, 3 or 4; and y is 0, 1, 2 or 3.

EP 2428512 discloses compounds of formula (G1) in which a¹ and a² separately represent an arylene group:

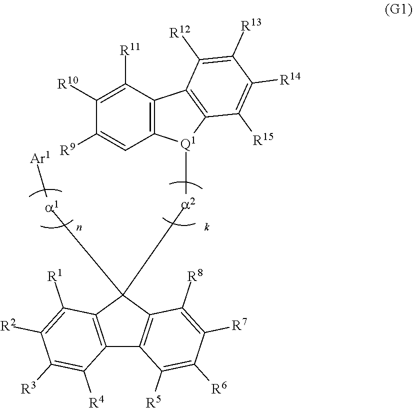

JP 2011/082238 discloses compounds of formula (1) in which at least one of Y₁ and Y₂ a group of formula (A) and Ar is a group of formula (B).

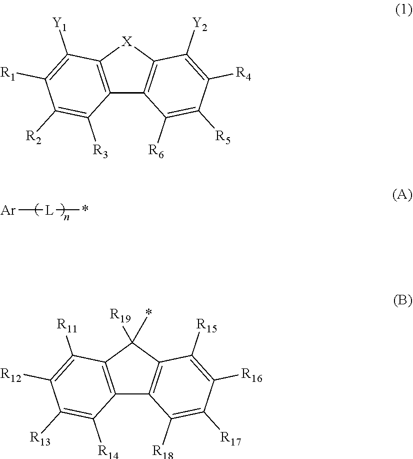

US 2012/0080667 discloses a composite material including an organic compound and an inorganic compound.

WO 2017/171376 discloses compounds of formula:

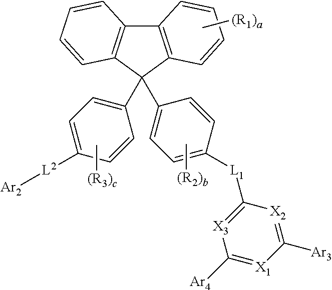

### SUMMARY

Phosphorescent emitters, in particular blue light emitting phosphorescent emitters, can suffer from relatively short lifetime. In the case of a white light-emitting OLED containing a blue phosphorescent emitter, the working life of the device may be limited by the lifetime of the blue phosphorescent emitter.

The present inventors have found that certain combinations of a host material and a phosphorescent emitter may provide OLEDs with long lifetime.

In some embodiments, there is provided a composition comprising a semiconducting host compound having a glass transition temperature (Tg) of less than 100° C. and a phosphorescent compound of formula (II):

M(L¹)p(L²)q (II)

For compound of formula (II), M is Ir (III) or Pt (II).

L¹ is a bidentate ligand of formula (III):

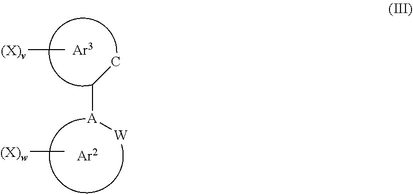

wherein:
Ar² is a 5-20 membered heteroaryl group; Ar³ is a C₆₋₂₀ arylene group or a 6-20 membered heteroaryl group; A is C or N; W is N if A is C and W is a carbene C atom if A is N; L² is a bidentate ligand which is different from L¹; p is at least 1; q is 0, 1 or 2; and each X independently comprises an aromatic or heteroaromatic group Ar⁵ which is unsubstituted or substituted with one or more substituents.
v and w are each independently 0 or 1 with the proviso that at least one of v and w is 1.

The sum of the number of rings comprised in the one or more X groups of formula (II) is at least 12 and at least 75% of the mass of each X is made up of the mass of the aromatic or heteroaromatic ring atoms of Ar⁵. “Aryl” and “heteroaryl” as used herein includes monocyclic and fused aryl and heteroaryl groups.

Each ring of (X)ᵥ or (X)_{w} may independently be an unfused ring which may be aromatic or non-aromatic, preferably aromatic; or an aromatic or non-aromatic ring fused to one or more aromatic or non-aromatic rings of a fused ring system.

Optionally, the semiconducting host compound has formula (I):

Ar²⁰—(Ar¹⁰)ᵤ—Ar³⁰ (I)

Ar¹⁰ independently is an arylene which is substituted or unsubstituted with one or more substituents and u is 1, 2, or 3; Ar²⁰ is a group of formula (Ia); and Ar³⁰ is a group of formula (Ib) or (Ic):

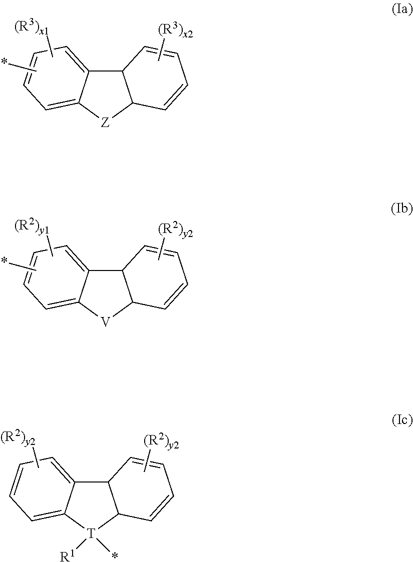

wherein Z is O or S; x₁ and y₁ are each independently 0, 1, 2 or 3; x₂ and y₂ are each independently 0, 1, 2, 3, or 4; V is O, S, —C(R⁹)₂— or —Si(R¹¹)₂—; T is C or Si; and R¹, R², R³, R⁹ and R¹¹ independently in each occurrence is a substituents.

In some embodiments, the compound of formula (I) is compound of formula (Ie) or (If):

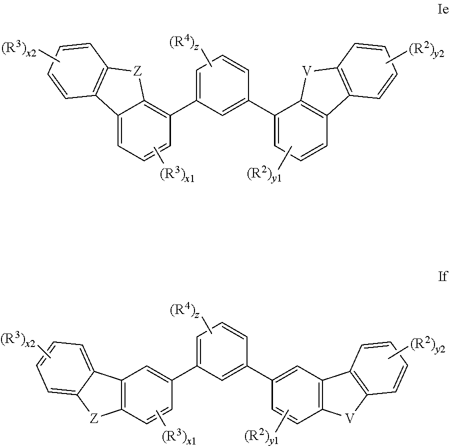

wherein Ar¹⁰ is a direct bond or an arylene or heteroarylene group.

In some embodiments, there is provided a formulation comprising a composition of a semiconducting host of formula (I) and a phosphorescent compound of formula (II) and one or more solvents.

In some embodiments, there is provided an organic light-emitting device comprising an anode, a cathode and a light-emitting layer between the anode and the cathode wherein the light-emitting layer comprises a composition of a semiconducting host of formula (I) and a phosphorescent compound of formula (II).

In some embodiments, there is provided a method of forming an organic light-emitting device comprising the step of forming the light-emitting layer over one of the anode and the cathode and forming the other of the anode and the cathode over the light-emitting layer

In some embodiments there is provided a composition comprising a compound of formula (IV) and a phosphorescent compound of formula (II):

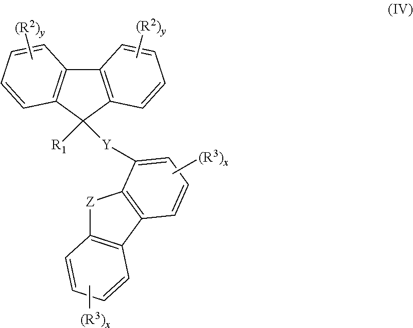

Wherein R¹, R², R³, x, y, Y and Z are as described anywhere herein.

The composition comprising the compound of formula (IV) and a phosphorescent compound of formula (II) may be provided in a formulation as described herein. This composition may be provided as the light-emitting layer of an OLED as described anywhere herein.

### DESCRIPTION OF THE DRAWINGS

The disclosed technology and accompanying figures describe some implementations of the disclosed technology.

FIG. 1 illustrates an OLED according to some embodiments;

FIG. 2 illustrates the electroluminescent spectra for a white OLED according to an embodiment and a comparative device which does not contain a phosphorescent emitter of formula (II);

FIG. 3 is a graph of luminance vs time for the white OLEDs of FIG. 2;

FIG. 4 is a graph of external quantum efficiency (EQE) vs. voltage for the white OLEDs of FIG. 2;

FIG. 5 illustrates the electroluminescent spectra for a white OLED according to an embodiment and a comparative device which does not contain a compound of formula (I);

FIG. 6 is a graph of luminance vs time for the white OLEDs of FIG. 5;

FIGS. 7A to 7D are graphs of photoluminescence vs time for films for compounds of formula (I) and phosphorescent emitters which are not compounds of formula (II);

FIG. 8 is a graph of photoluminescence vs time for films for compounds of formula (I) and a phosphorescent emitter formula (II);

FIGS. 9A and 9B are graphs of luminance vs time for an OLED device according to an embodiment which does not contain a phosphorescent emitter of formula (II);

FIGS. 10 and 11 are graphs of luminance vs time for an OLED device according to an embodiment which does contain a phosphorescent emitter formula (II).

FIG. 12 is a graph of luminance vs time for an OLED device according to an embodiment;

FIG. 13 illustrates the electroluminescent spectra according to an embodiment and a comparative device which does not contain a phosphorescent emitter of formula (II) for an OLED device of FIG. 12.

The drawings are not drawn to scale and have various viewpoints and perspectives. The drawings are some implementations and examples. Additionally, some components and/or operations may be separated into different blocks or combined into a single block for the purposes of discussion of some of the embodiments of the disclosed technology. Moreover, while the technology is amenable to various modifications and alternative forms, specific embodiments have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” References to a layer “over” another layer when used in this application means that the layers may be in direct contact or one or more intervening layers are may be present. References to a layer “on” another layer when used in this application means that the layers are in direct contact. Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. Details of the system may vary considerably in its specific implementation, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

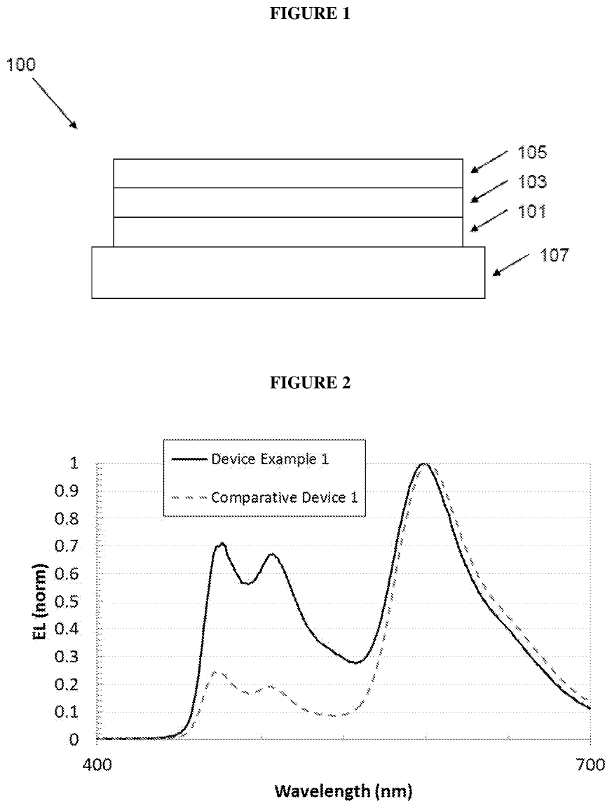

FIG. 1 illustrates an OLED 100 according to some embodiments comprising an anode 101, a cathode 105 and a light-emitting layer 103 between the anode and cathode. The device 100 is supported on a substrate 107, for example a glass or plastic substrate.

One or more further layers may be provided between the anode 101 and cathode 105, for example hole-transporting layers, electron transporting layers, hole blocking layers and electron blocking layers. The device may contain more than one light-emitting layer.

Preferred device structures include:

Anode/Hole-injection layer/Light-emitting layer/Cathode
Anode/Hole transporting layer/Light-emitting layer/Cathode
Anode/Hole-injection layer/Hole-transporting layer/Light-emitting layer/Cathode
Anode/Hole-injection layer/Hole-transporting layer/Light-emitting layer/Electron-transporting layer/Cathode.

Preferably, at least one of a hole-transporting layer and hole injection layer is present. Preferably, both a hole injection layer and hole-transporting layer are present.

Light-emitting layer 103 contains a host compound having a glass transition temperature (Tg) of less than 100° C., e.g. a compound of formula (I), doped with a light-emitting compound of formula (II). The light-emitting layer 103 may consist essentially of these materials or may contain one or more further materials, for example one or more charge-transporting materials or one or more further light-emitting materials. The lowest excited state triplet (T₁) energy level of the host is preferably the same as or higher than that of the light-emitting material in order to avoid quenching of luminescence from the light-emitting dopant.

The light-emitting layer 103 may contain one or more of a red light-emitting material, a green light-emitting material and a blue light-emitting material, at least one of the light-emitting materials being a compound of formula (II).

A blue emitting material may have a photoluminescent spectrum with a peak in the range of 400-490 nm, optionally 420-490 nm.

A green emitting material may have a photoluminescent spectrum with a peak in the range of more than 490 nm up to 580 nm, optionally more than 490 nm up to 540 nm

A red emitting material may optionally have a peak in its photoluminescent spectrum of more than 580 nm up to 630 nm, optionally 585-625 nm.

The photoluminescence spectrum of a light-emitting material may be measured by casting 5 wt % of the material in a polystyrene film onto a quartz substrate and measuring in a nitrogen environment using apparatus C9920-02 supplied by Hamamatsu.

The host:compound of formula (II) weight ratio is preferably in the range of about 99.9:0.1-55:45.

The host preferably has a T₁ of greater than 2.8 eV, preferably greater than 3.0 eV.

Triplet energy levels of host materials and compounds of formula (II) may be measured from the energy onset of the phosphorescence spectrum measured by low temperature phosphorescence spectroscopy (Y. V. Romaovskii et al, Physical Review Letters, 2000, 85 (5), p 1027, A. van Dijken et al, Journal of the American Chemical Society, 2004, 126, p 7718).

The host preferably has a HOMO level of at least 5.8 eV from vacuum level, preferably at least 5.9 eV from vacuum level. HOMO and LUMO levels as given herein are as measured by square wave voltammetry.

Preferably, the compound of formula (II) has a HOMO level at least 0.1 eV closer to vacuum than the host, optionally at least 0.5 eV closer to vacuum.

In a preferred embodiment, the compound of formula (II) is a blue phosphorescent light-emitting material.

Light-emitting layer 103 may be unpatterned, or may be patterned to form discrete pixels. Each pixel may be further divided into subpixels. The light-emitting layer may contain a single light-emitting material, for example for a monochrome display or other monochrome device, or may contain materials emitting different colours, in particular red, green and blue light-emitting materials for a full-colour display.

The OLED may contain more than one light-emitting material, for example a mixture of light-emitting materials that together provide white light emission.

A white-emitting OLED may contain a single, white-emitting layer containing a light-emitting composition, or may contain two or more layers that emit different colours which, in combination, produce white light and wherein at least one of the light emitting layers comprises a composition as described herein.

The light emitted from a white-emitting OLED may have CIE x coordinate equivalent to that emitted by a black body at a temperature in the range of 2500-9000K and a CIE y coordinate within 0.05 or 0.025 of the CIE y co-ordinate of said light emitted by a black body, optionally a CIE x coordinate equivalent to that emitted by a black body at a temperature in the range of 2700-6000K.

Host

The host has a glass transition temperature of less than 100° C.

Preferably, the host is a compound of formula (I):

Ar²⁰—(Ar¹⁰)ᵤ—Ar³⁰ (I)

u is 1, 2, or 3.
Ar¹⁰ independently in each occurrence is an arylene. Ar¹⁰ is optionally selected from C₆₋₂₀ arylenes.
Ar¹⁰ may be substituted or unsubstituted with one or more groups R⁴ wherein R⁴ in each occurrence is independently a substituent. If present, substituents R⁴ are optionally selected from branched, linear or cyclic C₁₋₂₀ alkyl wherein one or more non-adjacent C atoms may be replaced with O, S, CO or COO.
Ar¹⁰ is preferably phenylene that may be substituted or unsubstituted with one or more substituents R⁴. In some preferred embodiments, Ar¹⁰ may be an ortho-linked group of formula (Xa), a para-linked group of formula (Xb) or a meta-linked group of formula (Xc). The extent of conjugation across a meta-linked phenylene group Ar¹⁰ may be limited as compared to a para-linked phenylene group Ar¹⁰.

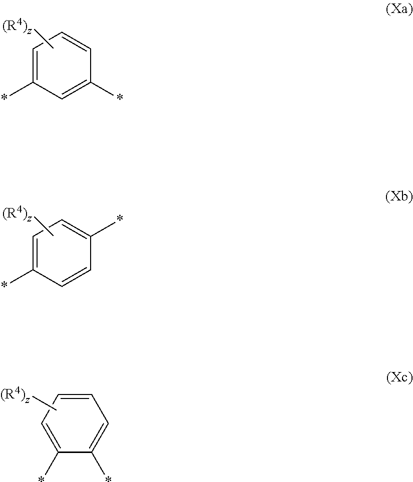

When u is 2 or 3, Ar¹⁰ in each occurrence, may independently be the same or different, and each Ar¹⁰ may differ in its points of attachment to an adjacent Ar¹⁰ group or Ar²⁰ or Ar³⁰.

—(Ar¹⁰)ᵤ— may be selected, without limitation, from:

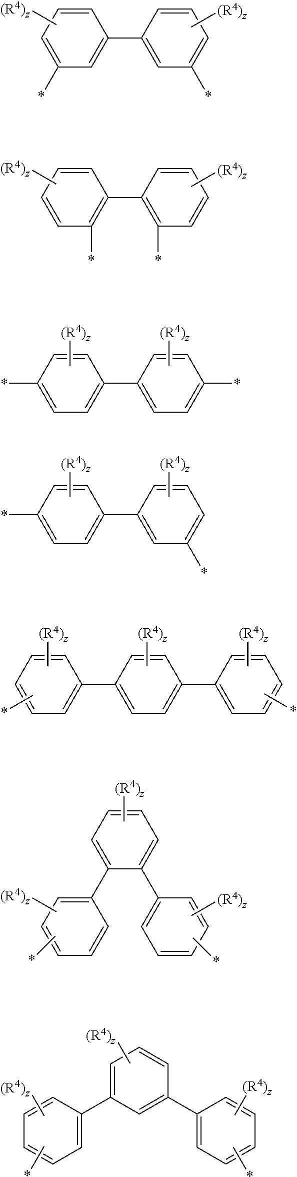

wherein
R⁴ is independently a substituent; and
z is 0, 1, 2, 3 or 4.

Preferably, —(Ar¹⁰)ᵤ— is selected from:

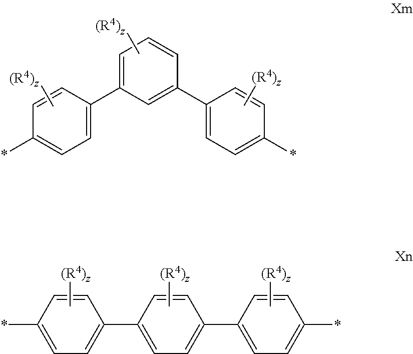

Ar²⁰ is a group of formula (Xd):

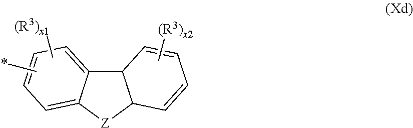

wherein

### Z is O or S;

R³ is independently a substituent;
x₁ is 1, or 2 or 3; and
x₂ is 1, or 2, or 3, or 4.

In some preferred embodiments, compound of formula (Xd) is a group of formula (Xe) or (XI):

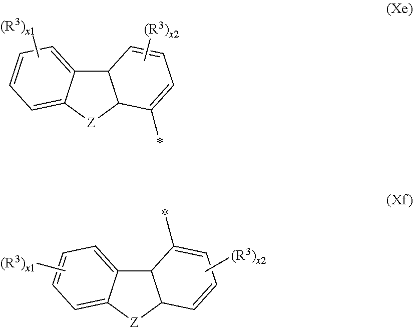

Ar³⁰ is a group of formula (Xg) or (Xh):

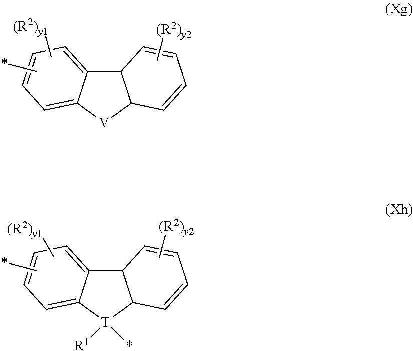

wherein
V is O, S, —C(R⁹)₂— or —Si(Z¹¹)₂—;

### T is C or Si;

R¹ and R² are each independently a substituent;
y₁ is 1, or 2 or 3; and
y₂ is 1, or 2, or 3, or 4.

In some embodiments, the host is compound of formula (IV):

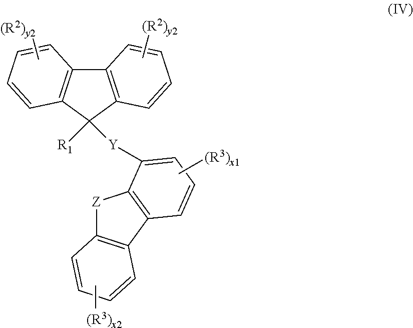

wherein:

### Z is O or S;

R¹, R² and R³ are each independently a substituent;
x₁ is 0, 1, 2, or 3;
x₂ is 0, 1, 2, 3 or 4;
y₂ is 0, 1, 2, 3 or 4; and
Y is a direct bond or an arylene or heteroarylene group Ar¹.
Ar¹ is optionally selected from C₆₋₂₀ arylenes and 5-20 membered heteroarylenes.
Ar¹ may be selected from arylene groups Ar¹⁰. In this case, it will be understood that the compound of formula (IV) is a compound of formula (I).

Optionally, R¹ of formula (IV) or formula (Xh) is selected from the group consisting of linear, branched or cyclic C₁₋₂₀ alkyl wherein one or more non-adjacent, non-terminal C atoms may be replaced with O, S, CO or COO and one or more H atoms may be replaced with F; and a group of formula —(Ar⁴)ₙ wherein n is at least 1, optionally 1-3; and Ar⁴ in each occurrence is independently selected from aryl or heteroaryl which is unsubstituted or substituted with one or more substituents.

By “non-terminal C atom” of an alkyl group is meant a C atom of an alkyl group other than the methyl group of a linear alkyl chain or the methyl groups of a branched alkyl chain.

Ar⁴ is preferably C₆₋₂₀ aryl or 5-20 membered heteroaryl, and each Ar⁴ is independently unsubstituted or substituted with one or more substituents, optionally one or more C₁₋₁₂ alkyl groups wherein one or more non-adjacent, non-terminal C atoms may be replaced with O, S, CO or COO and one or more H atoms may be replaced with F.

In some preferred embodiments, R¹ of formula (IV) or formula (Xh) is bound to the 9-position of the fluorene unit through a sp³ hybridised carbon atom. According to these embodiments, R¹ is preferably a linear, branched or cyclic C₁₋₂₀ alkyl group, more preferably methyl.

In some preferred embodiments, R¹ of formula (IV) is a group of formula (Xi):

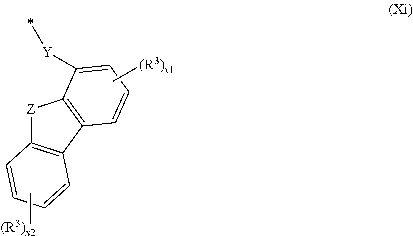

wherein R³, Y, Z, and x are as described above, and * is a point of attachment to the fluorene group of formula (I). In the case where R¹ is a group of formula (Xi), each R³, Y, Z, and x of the compound of formula (I) may independently be the same or different.

If present, R² and R³ of formula (I) or formula (IV) are preferably in each occurrence independently selected from linear, branched or cyclic C₁₋₁₂ alkyl; and aryl or heteroaryl, preferably C₆₋₂₀ aryl or 5-20 membered heteroaryl, which may be unsubstituted or substituted with one or more substituents, optionally one or more C₁₋₁₂ alkyl groups.

Preferably, an aryl or heteroaryl group R² or R³ is phenyl that may be unsubstituted or substituted with one or more substituents.

Each x is preferably 0.

Each y is preferably 0.

Preferably the compound of formula (I) or formula (IV) is selected from:

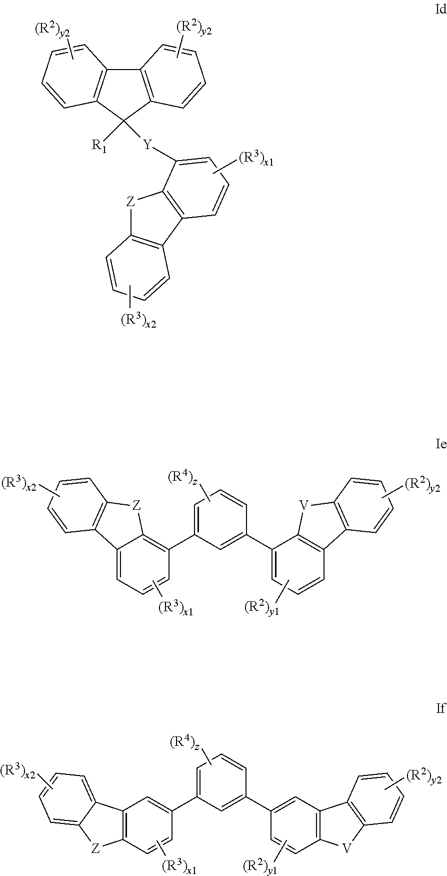

wherein V, Y, Z, R¹, R², R³, R⁴, x₁, x₂, y₁, y₂ and z are as previously defined.

Exemplary compounds of formula (I) and formula (IV) are:

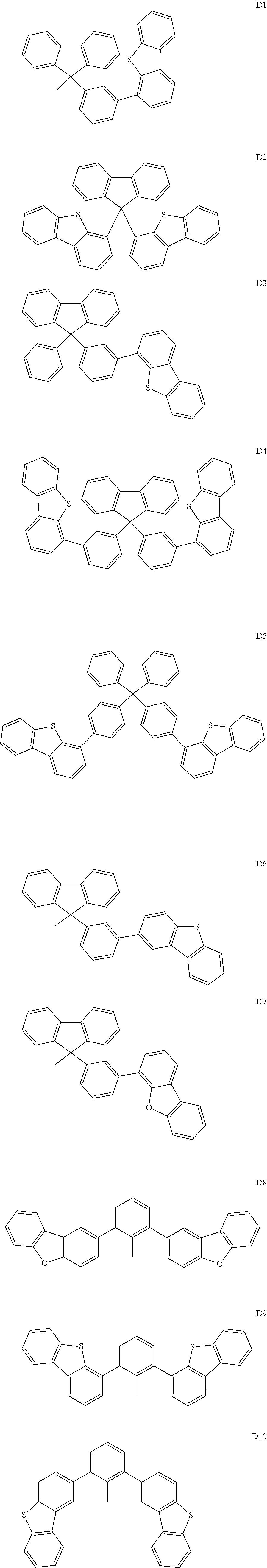

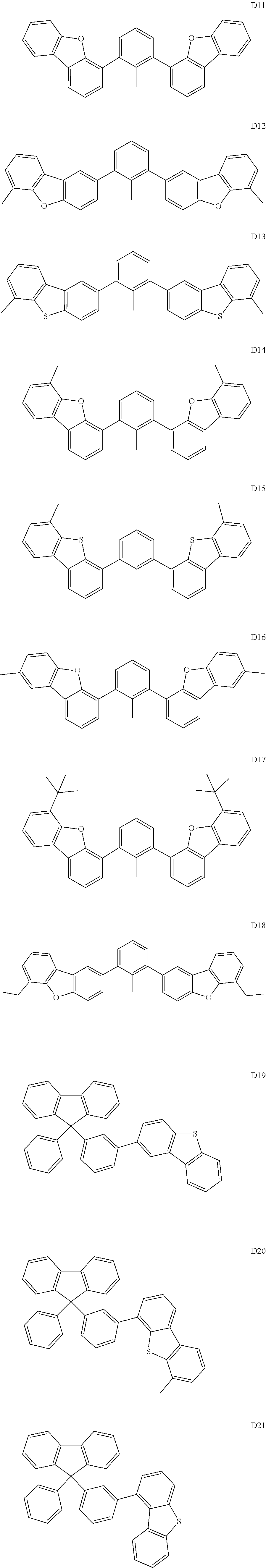

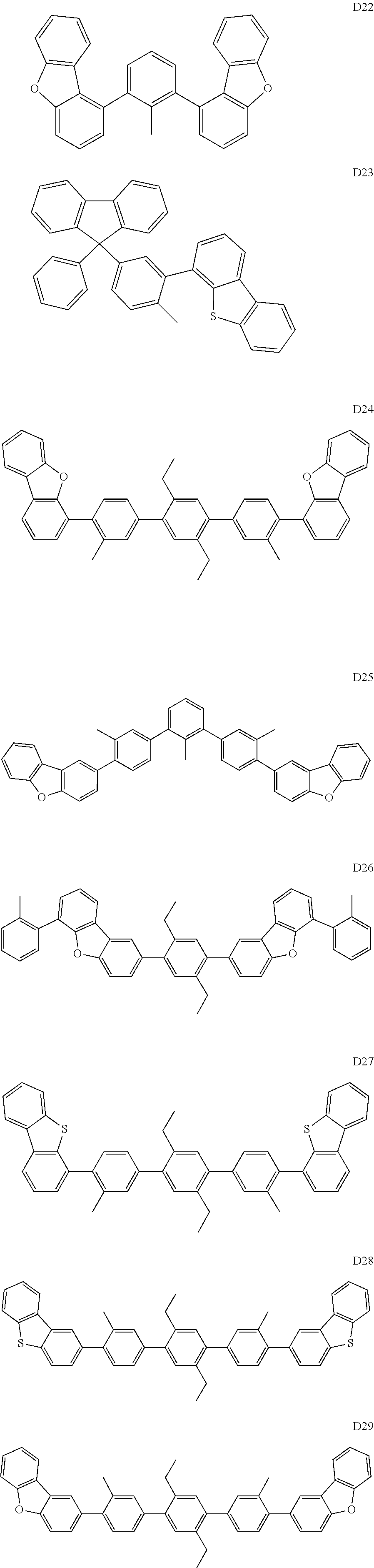

| | Tg |
|---|---|
| D1 | 62 |
| D3 | 92 |
| D6 | 62 |
| D7 | 52 |
| D8 | 54 |
| D9 | 84 |
| D10 | 87 |
| D11 | 80 |
| D12 | 80 |
| D13 | 89 |
| D14 | 79 |
| D15 | 97 |
| D16 | 89 |
| D17 | 82 |
| D18 | 59 |
| D19 | 93 |
| D20 | 96 |
| D21 | 89 |
| D22 | 55 |
| D23 | 97 |
| D24 | 89 |
| D25 | 83 |
| D26 | 93 |
| D27 | 98 |
| D28 | 89 |
| D29 | 71 |

Compounds of Formula (II)

The compound of formula (II) is:

M(L¹)p(L²)q (II)

wherein:

### M is Ir (III) or Pt (II);

L¹ is a bidentate ligand of formula (III):

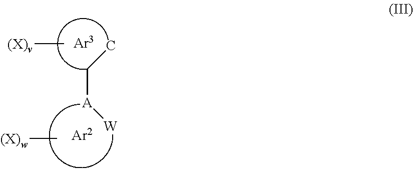

wherein:
Ar² is a 5-20 membered heteroaryl group;
Ar³ is a C₆₋₂₀ arylene group or a 5-20 membered heteroaryl group;

### A is C or N;

W is N if A is C and W is a carbene C atom if A is N;
p is at least 1;
q is 0, 1 or 2;
each X independently comprises an aromatic or heteroaromatic group Ar⁵ which is unsubstituted or substituted with one or more substituents;
v and w are each independently 0 or 1 with the proviso that at least one of v and w is 1;
the sum of the number of rings comprised in the one or more X groups of formula (I) is at least 12; and
at least 75% of the mass of each X is made up of the mass of the aromatic or heteroaromatic ring atoms of Ar⁵;
and wherein each L² is independently a ligand different from L¹.

Each X group comprises or consists of an aromatic or heteroaromatic group Ar⁵ which is unsubstituted or substituted with one or more substituents. If X comprises 2 or more Ar⁵ groups then Ar⁵ in each occurrence may be the same or different.

In some embodiments, each Ar⁵ is directly bound to at least one other Ar⁵.

The linked Ar⁵ groups may form a linear or branched chain of Ar⁵ groups of formula —(Ar⁵)ₘ in which m is at least 2.

A linear chain of Ar⁵ groups may have formula —(Ar⁵)ᵤ—R¹⁸ wherein each Ar⁵ is independently an arylene or heteroarylene group which is unsubstituted or substituted with one or more substituents and R¹⁸ is H or a substituent and u is at least 2.

Optionally, u is at least 3, at least 4 or at least 5.

Each Ar⁵ may independently be unsubstituted or substituted with one or more substituents. Substituents of Ar⁵ may be selected from R⁶, wherein R⁶ in each occurrence is independently selected from F, CN, NO₂, and C₁₋₁₂ alkyl wherein one or more non-adjacent, non-terminal C atoms may be replaced with O, S, CO or COO and one or more H atoms may be replaced with F.

Optionally, if R¹⁸ is a substituent it is selected from the group R⁶.

A branched chain of Ar⁵ groups comprises three or more Ar⁵ groups directly linked to one another wherein at least one of the Ar⁵ groups is a branching Ar⁵ group directly linked to at least two other Ar⁵ groups and wherein each Ar⁵ group is independently unsubstituted or substituted with one or more substituents.

In some embodiments, X is a group of formula (VIII):

—Ar⁵-[(L-Ar⁵)ₛ]ₜ (VIII)

wherein L is a divalent linking group selected from 0, S or NR¹⁷ wherein R¹⁷ in each occurrence is C₁₋₁₂ alkyl; s is at least 1; and t is at least 1.

A group of formula (VIII) may be arranged as a linear chain (t=1) or a branched chain (t=at least 2, optionally 2 or 3).

Optionally, s is at least 2, at least 3 or at least 4.

At least 75% of the mass of each X, optionally at least 80%, at least 85% or at least 90%, is made up of the mass of the aromatic or heteroaromatic ring atoms of Ar⁵. Substituents of Ar⁵, such as R⁶, (if any) and divalent linking groups L (if any) may be selected accordingly.

Each Ar⁵ is independently a monocyclic aromatic or heteroaromatic ring or a fused aromatic or heteroaromatic group, preferably a C₆₋₂₀ aromatic group or a 5-20 membered heteroaromatic group. Preferred Ar⁵ groups are benzene (one ring); fluorene; dibenzothiophene; dibenzofuran; and carbazole (each three rings), each of which is independently unsubstituted or substituted with one or more substituents.

Exemplary groups X are illustrated below, wherein each aromatic or heteroaromatic group may independently be unsubstituted or substituted with one or more substituents, preferably one or more C₁₋₁₂ alkyl groups:

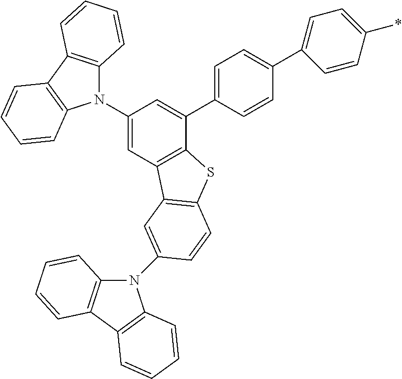 [SMILES: *c1ccc(-c2ccc(-c3cc(-n4c5ccccc5c5ccccc54)cc4c3sc3ccc(-n5c6ccccc6c6ccccc65)cc34)cc2)cc1]

number of rings: 11.

It will be understood that the sum of the number of rings comprised in the one or more X groups of formula (II) is:

p×[the number of rings in (X)v+the number of rings in (X)w].

If p is 1 and only one of v and w is 1 then the compound of formula (II) comprises only one X group which comprises more than 12 rings.

If p is 2 or 3 and/or if both of v and w are 1 then each X may comprise one or more rings with the proviso that the sum of the rings of the X groups is greater than 12.

In some embodiments, the sum of the number of rings comprised in the one or more X groups of formula (II) is at least 20, optionally at least 25, optionally at least 30. Optionally, the sum is no more than 50, optionally no more than 45.

In some embodiments, each X group comprises at least 5, optionally at least 10, rings.

In some embodiments, each X group comprises no more than 25 rings, optionally no more than 20 rings, optionally no more than 15 rings.

In some embodiments, v is 0 and w is 1.

In some embodiments, w is 0 and v is 1.

In some embodiments, v and w are each 1.

If v=1 then the group X may be the only substituent of Ar³, or Ar³ may be substituted with one or more further substituents.

If w=1 then the group X may be the only substituent of Ar², or Ar² may be substituted with one or more further substituents.

Further substituents of Ar² and Ar³, where present, are optionally selected from R¹⁴, wherein R¹⁴ in each occurrence is independently selected from the group consisting of: D; F; CN; NO₂; and C₁₋₂₀ alkyl wherein one or more non-adjacent C atoms may be replaced with O, S, CO or COO and one or more H atoms may be replaced with F.

It will be understood that the C atom of Ar³ illustrated in Formula (III) is a carbanion.

In some preferred embodiments, Ar³ is phenyl or naphthyl.

In some preferred embodiments, A is C, W is N and Ar² is a 5, 6 or 10 membered heterocyclic group having C and N ring atoms, preferably a diazole; a triazole; pyridyl, quinolinyl, or isoquinolinyl, each of which may or may not be substituted with a substituent R¹⁶.

In some embodiments, the compound of formula (II) has formula (IIa):

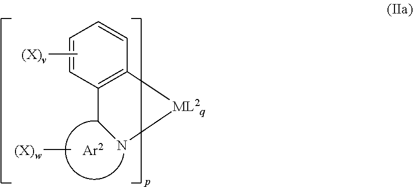

Optionally, the compound of formula (II) is selected from:

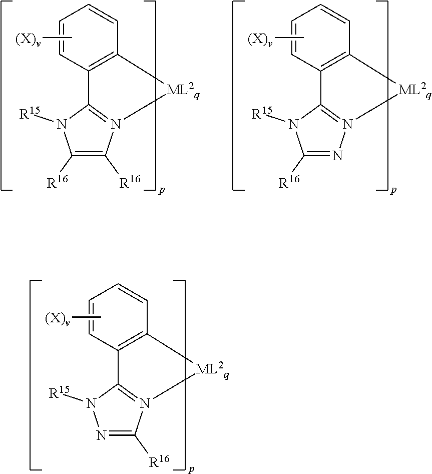

wherein R¹⁵ in each occurrence is selected from the group consisting of X and C₁₋₂₀ alkyl; R¹⁶ in each occurrence is H or R¹⁵; and, if v is 0, one of R¹⁵ and R¹⁶ is X.

In some preferred embodiments, v is 0 and one of R¹⁵ and R¹⁶ is X.

In some preferred embodiments, v is 0, R¹⁵ is a group of formula X and R¹⁶ is H or C₁₋₁₂ alkyl.

In some embodiments, A is N and W is a carbene carbon atom. According to these embodiments, the compound of formula (II) may have formula (IIb):

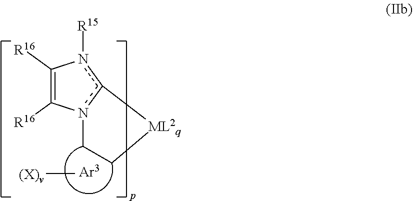

wherein R¹⁵ and R¹⁶ are described above and wherein the two R¹⁶ groups may be linked to form a ring; one of R¹⁵ and, if w=0, R¹⁶ is a group of formula X or a ring formed by linkage of the two groups R¹⁶ is substituted with a group of formula X.

Where M is Ir(III), it is preferred that p is 3 and q is 0, p is 2 and q is 1, or p is 1 and q is 2.

Where M is Pt(II), it is preferred that p is 2 and q is 0, or p and q are each 1.

L², if present, is preferably a bidentate ligand, optionally a bidentate ligand selected from:
a ligand of formula Ar²—Ar³ as described with reference to formula (II) except that the ligand is not substituted with any substituents X; and
N, N-, N,O- or O,O-bidentate ligands, for example a diketonate such as acac.

A ligand L² of formula Ar²—Ar³ is optionally substituted with one or more substituent selected from R¹⁴ as described above.

Preferably, M is Ir and either:

p is 3 and q is 0;
p is 2 and p is 1; or
p is 1 and q is 2.

Preferably, p is 2 or 3.

Charge Transporting and Charge Blocking Layers

A device containing a light-emitting layer containing a composition as described herein may have charge-transporting and/or charge blocking layers.

A hole transporting layer may be provided between the anode and the light-emitting layer or layers of an OLED. An electron transporting layer may be provided between the cathode and the light-emitting layer or layers.

An electron blocking layer may be provided between the anode and the light-emitting layer(s) and a hole blocking layer may be provided between the cathode and the light-emitting layer(s). Charge-transporting and charge-blocking layers may be used in combination. Depending on the HOMO and LUMO levels of the material or materials in a layer, a single layer may both transport one of holes and electrons and block the other of holes and electrons.

If present, a hole transporting layer located between the anode and the light-emitting layer(s) preferably has a material having a HOMO level of less than or equal to 5.5 eV, more preferably around 4.8-5.5 eV or 4.9-5.3 eV as measured by square wave voltammetry. The HOMO level of the material in the hole transport layer may be selected so as to be within 0.2 eV, optionally within 0.1 eV of the light-emitting material of the light-emitting layer.

A hole-transporting layer may contain polymeric or non-polymeric charge-transporting materials. Exemplary hole-transporting materials contain arylamine groups.

A hole transporting layer may contain a homopolymer or copolymer comprising a repeat unit of formula (VII):

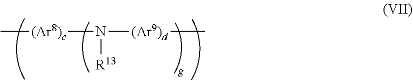

wherein Ar⁸ and Ar⁹ in each occurrence are independently selected from substituted or unsubstituted aryl or heteroaryl, g is greater than or equal to 1, preferably 1 or 2, R¹³ is H or a substituent, preferably a substituent, and c and d are each independently 1, 2 or 3.

R¹³, which may be the same or different in each occurrence when g>1, is preferably selected from the group consisting of alkyl, for example C₁₋₂₀ alkyl, Ar¹¹, a branched or linear chain of Ar¹¹ groups, or a crosslinkable unit that is bound directly to the N atom of formula (VIII) or spaced apart therefrom by a spacer group, wherein Ar¹¹ in each occurrence is independently optionally substituted aryl or heteroaryl. Exemplary spacer groups are C₁₋₂₀ alkyl, phenyl and phenyl-C₁₋₂₀ alkyl.

Any of Ar⁸, Ar⁹ and, if present, Ar¹¹ in the repeat unit of Formula (VII) may be linked by a direct bond or a divalent linking atom or group to another of Ar⁸, Ar⁹ and Ar¹¹. Preferred divalent linking atoms and groups include 0, S; substituted N; and substituted C.

Any of Ar⁸, Ar⁹ and, if present, Ar¹¹ may be substituted with one or more substituents. Exemplary substituents are substituents R¹⁰, wherein each R¹⁰ may independently be selected from the group consisting of:
substituted or unsubstituted alkyl, optionally C₁₋₂₀ alkyl, wherein one or more non-adjacent C atoms may be replaced with optionally substituted aryl or heteroaryl, O, S, substituted N, C═O or —COO— and one or more H atoms may be replaced with F; and
a crosslinkable group attached directly to Ar⁸, Ar⁹ or Ar¹¹ or spaced apart therefrom by a spacer group, for example a group comprising a double bond such and a vinyl or acrylate group, or a benzocyclobutane group

Preferred repeat units of formula (VII) have formulae 1-3:

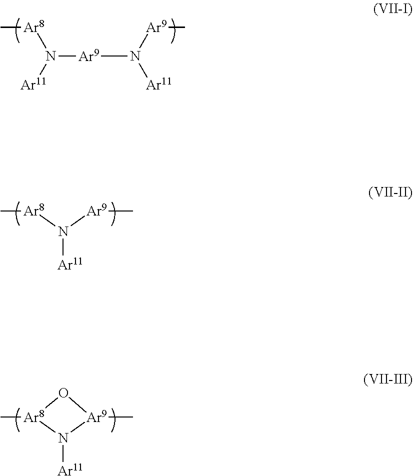

In one preferred arrangement, R¹³ is Ar¹¹ and each of Ar⁸, Ar⁹ and Ar¹¹ are independently and optionally substituted with one or more C₁₋₂₀ alkyl groups. Ar⁸, Ar⁹ and Ar¹¹ are preferably phenyl.

In another preferred arrangement, the central Ar⁹ group of formula (VII-1) linked to two N atoms is a polycyclic aromatic that may be unsubstituted or substituted with one or more substituents R¹⁰. Exemplary polycyclic aromatic groups are naphthalene, perylene, anthracene and fluorene.

In another preferred arrangement, Ar⁸ and Ar⁹ are phenyl, each of which may be substituted with one or more C₁₋₂₀ alkyl groups, and R¹³ is —(Ar¹¹), wherein r is at least 2 and wherein the group —(Ar¹¹), forms a linear or branched chain of aromatic or heteroaromatic groups, for example 3,5-diphenylbenzene wherein each phenyl may be substituted with one or more C₁₋₂₀ alkyl groups. In another preferred arrangement, c, d and g are each 1 and Ar⁸ and Ar⁹ are phenyl linked by an oxygen atom to form a phenoxazine ring.

A hole-transporting polymer containing repeat units of formula (VII) may be a copolymer containing one or more further repeat units. Exemplary further repeat units include arylene repeat units, each of which may be unsubstituted or substituted with one or more substituents.

Exemplary arylene repeat units include without limitation, fluorene, phenylene, naphthalene, anthracene, indenofluorene, phenanthrene and dihydrophenanthrene repeat units, each of which may be unsubstituted or substituted with one or more substituents.

Substituents of arylene repeat units, if present, may be selected from C₁₋₄₀ hydrocarbyl, preferably C₁₋₂₀ alkyl; phenyl which may be unsubstituted or substituted with one or more C₁₋₁₀ alkyl groups; and crosslinkable hydrocarbyl groups, for example C₁₋₄₀ hydrocarbyl groups comprising benzocyclobutene or vinylene groups.

Phenylene repeat units may be 1,4-linked phenylene repeat units that may be unsubstituted or substituted with 1, 2, 3 or 4 substituents. Fluorene repeat units may be 2,7-linked fluorene repeat units.

Fluorene repeat units preferably have two substituents in the 9-position thereof. Aromatic carbon atoms of fluorene repeat units may each independently be unsubstituted or substituted with a substituent.

If present, an electron transporting layer located between the light-emitting layers and cathode preferably has a LUMO level of around 1.8-2.7 eV as measured by square wave voltammetry. An electron-transporting layer may have a thickness in the range of about 5-50 nm

A charge-transporting layer or charge-blocking layer may be crosslinked, particularly if a layer overlying that charge-transporting or charge-blocking layer is deposited from a solution. The crosslinkable group used for this crosslinking may be a crosslinkable group comprising a reactive double bond such and a vinyl or acrylate group, or a benzocyclobutane group. The crosslinkable group may be provided as a substituent of, or may be mixed with, a charge-transporting or charge-blocking material used to form the charge-transporting or charge-blocking layer.

A charge-transporting layer adjacent to a light-emitting layer containing a composition as described preferably contains a charge-transporting material having a lowest triplet excited state (T₁) excited state that is no more than 0.1 eV lower than, preferably the same as or higher than, the T₁ excited state energy level of the phosphorescent light-emitting material(s) of the light-emitting layer in order to avoid quenching of triplet excitons.

A charge-transporting layer as described herein may be non-emissive, or may contain a light-emitting material such that the layer is a charge transporting light-emitting layer. If the charge-transporting layer is a polymer then a light-emitting dopant may be provided as a side-group of the polymer, a repeat unit in a backbone of the polymer, or an end group of the polymer. Optionally, a hole-transporting polymer as described herein comprises a phosphorescent polymer in a side-group of the polymer, in a repeat unit in a backbone of the polymer, or as an end group of the polymer.

The polystyrene-equivalent number-average molecular weight (Mn) measured by gel permeation chromatography of the polymers described herein may be in the range of about 1×10³ to 1×10⁸, and preferably 1×10⁴ to 5×10⁶. The polystyrene-equivalent weight-average molecular weight (Mw) of the polymers described herein may be 1×10³ to 1×10⁸, and preferably 1×10⁴ to 1×10⁷.

Polymers as described herein are suitably amorphous.

Hole Injection Layers

A conductive hole injection layer, which may be formed from a conductive organic or inorganic material, may be provided between the anode 101 and the light-emitting layer 103 of an OLED as illustrated in FIG. 1 to assist hole injection from the anode into the layer or layers of semiconducting polymer. Examples of doped organic hole injection materials include optionally substituted, doped poly(ethylene dioxythiophene) (PEDOT), in particular PEDOT doped with a charge-balancing polyacid such as polystyrene sulfonate (PSS) as disclosed in EP 0901176 and EP 0947123, polyacrylic acid or a fluorinated sulfonic acid, for example Nafion®; polyaniline as disclosed in U.S. Pat. Nos. 5,723,873 and 5,798,170; and optionally substituted polythiophene or poly(thienothiophene). Examples of conductive inorganic materials include transition metal oxides such as VOx, MoOx and RuOx as disclosed in Journal of Physics D: Applied Physics (1996), 29(11), 2750-2753.

Cathode

The cathode 105 is selected from materials that have a work function allowing injection of electrons into the light-emitting layer of the OLED. Other factors influence the selection of the cathode such as the possibility of adverse interactions between the cathode and the light-emitting material. The cathode may consist of a single material such as a layer of aluminium. Alternatively, it may comprise a plurality of conductive materials such as metals, for example a bilayer of a low work function material and a high work function material such as calcium and aluminium, for example as disclosed in WO 98/10621. The cathode may comprise elemental barium, for example as disclosed in WO 98/57381, Appl. Phys. Lett. 2002, 81(4), 634 and WO 02/84759. The cathode may comprise a thin (e.g. 1-5 nm) layer of metal compound, in particular an oxide or fluoride of an alkali or alkali earth metal, between the organic layers of the device and one or more conductive cathode layers to assist electron injection, for example lithium fluoride as disclosed in WO 00/48258; barium fluoride as disclosed in Appl. Phys. Lett. 2001, 79(5), 2001; and barium oxide. In order to provide efficient injection of electrons into the device, the cathode preferably has a work function of less than 3.5 eV, more preferably less than 3.2 eV, most preferably less than 3 eV. Work functions of metals can be found in, for example, Michaelson, J. Appl. Phys. 48(11), 4729, 1977.

The cathode may be opaque or transparent. Transparent cathodes are particularly advantageous for active matrix devices because emission through a transparent anode in such devices is at least partially blocked by drive circuitry located underneath the emissive pixels. A transparent cathode comprises a layer of an electron injecting material that is sufficiently thin to be transparent. Typically, the lateral conductivity of this layer will be low as a result of its thinness. In this case, the layer of electron injecting material is used in combination with a thicker layer of transparent conducting material such as indium tin oxide.

It will be appreciated that a transparent cathode device need not have a transparent anode (unless, of course, a fully transparent device is desired), and so the transparent anode used for bottom-emitting devices may be replaced or supplemented with a layer of reflective material such as a layer of aluminium. Examples of transparent cathode devices are disclosed in, for example, GB 2348316.

Encapsulation

Organic optoelectronic devices tend to be sensitive to moisture and oxygen. Accordingly, the substrate preferably has good barrier properties for prevention of ingress of moisture and oxygen into the device. The substrate is commonly glass, however alternative substrates may be used, in particular where flexibility of the device is desirable. For example, the substrate may comprise one or more plastic layers, for example a substrate of alternating plastic and dielectric barrier layers or a laminate of thin glass and plastic.

The device may be encapsulated with an encapsulant (not shown) to prevent ingress of moisture and oxygen. Suitable encapsulants include a sheet of glass, films having suitable barrier properties such as silicon dioxide, silicon monoxide, silicon nitride or alternating stacks of polymer and dielectric or an airtight container. In the case of a transparent cathode device, a transparent encapsulating layer such as silicon monoxide or silicon dioxide may be deposited to micron levels of thickness, although in one preferred embodiment the thickness of such a layer is in the range of 20-300 nm. A getter material for absorption of any atmospheric moisture and/or oxygen that may permeate through the substrate or encapsulant may be disposed between the substrate and the encapsulant.

Formulation Processing

A formulation suitable for forming a charge-transporting or light-emitting layer may be formed from a composition as described herein and one or more suitable solvents.

The formulation may be a solution of the composition and any other components in the one or more solvents, or may be a dispersion in the one or more solvents in which one or more components are not dissolved. Preferably, the formulation is a solution.

Solvents suitable for dissolving compositions as described herein are benzenes substituted with one or more C₁₋₁₀ alkyl or C₁₋₁₀ alkoxy groups, for example toluene, xylenes and methylanisoles.

Particularly preferred solution deposition techniques including printing and coating techniques such spin-coating, inkjet printing and slot-die coating.

Spin-coating is particularly suitable for devices wherein patterning of the light-emitting layer is unnecessary—for example for lighting applications or simple monochrome segmented displays.

Inkjet printing is particularly suitable for high information content displays, in particular full colour displays. A device may be inkjet printed by providing a patterned layer over the first electrode and defining wells for printing of one colour (in the case of a monochrome device) or multiple colours (in the case of a multicolour, in particular full colour device). The patterned layer is typically a layer of photoresist that is patterned to define wells as described in, for example, EP 0880303.

As an alternative to wells, the ink may be printed into channels defined within a patterned layer. In particular, the photoresist may be patterned to form channels which, unlike wells, extend over a plurality of pixels and which may be closed or open at the channel ends.

Other solution deposition techniques include dip-coating, roll printing and screen printing.

### EXAMPLES

### Host 1 (D1)

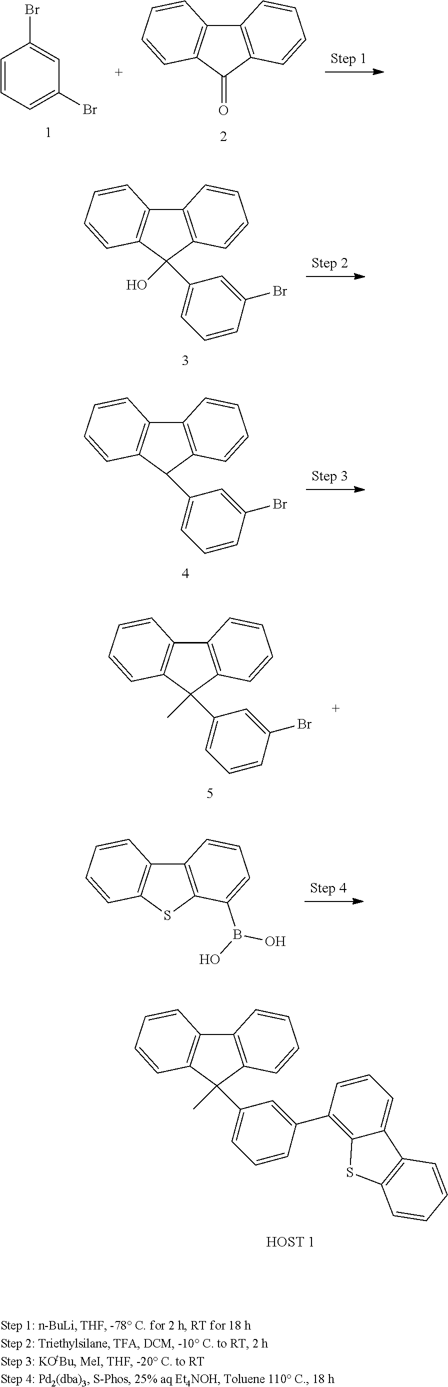

### Step 1—Synthesis of Intermediate 3

To a solution of 1,3-dibromobenzene (288 g, 1.22 mol) in THF (2 L) at −78° C., was added, 2.5M n-BuLi in hexane (443 mL, 1.11 mol). After stirring at −78° C. for 2 h, 9-fluorenone (200 g, 1.11 mol) in THF (500 mL) was slowly added, the reaction mixture was allowed to warm to room temperature, stirred for 18 h and then quenched with saturated NH₄Cl solution (200 mL) and extracted with EtOAc (3×1 L). The combined organic phase was washed with water (1000 mL), brine (500 mL), dried over sodium sulphate and concentrated. The residue showed ˜60% Intermediate 3 and was used in the next step without further purification.

### Step 2—Synthesis of Intermediate 4

A solution of Intermediate 3 (˜60% pure, 420 g, 0.77 mol) and triethyl silane (186 mL, 1.16 mol) in anhydrous DCM (3 L), under N₂, was cooled to −10° C. and stirred for 0.5 h. Trifluoroacetic acid (175 mL, 2.31 mol) was slowly added and the reaction mixture was stirred at room temperature for 2 h. The reaction mixture was quenched with water (300 mL) and the organic phase was washed with water (500 mL), brine (500 mL), dried over sodium sulphate and concentrated. The crude product was purified by silica column chromatography (3 to 4% EtOAc in hexane), triturated with methanol and recrystallized from hot acetonitrile to give 195 g of intermediate 4 [HPLC:_97.02%].

### Step 3—Synthesis of Intermediate 5

Intermediate 4 (195 g, 0.61 mol) was dissolved in dry THF (1.8 L) and degassed with N₂ for an hour and then cooled to −20° C. A degassed solution of KO′Bu (68.1 g, 0.61 mol) in THF (1.2 L) and MeI (37.9 mL, 0.61 mol) was added to dropwise to Intermediate 4.

The reaction mixture was slowly allowed to warm to room temperature, stirred for 18 h, quenched with NH₄Cl solution (500 mL) and extracted with EtOAc (3×1 L). The combined organic phases were washed with water (1 L), brine (500 mL), dried over sodium sulphate and concentrated (210 g) and purified by silica column chromatography (5 to 6% EtOAc in hexane) followed by recrystallization from hot methanol to give 155 g of Intermediate 5 [HPLC: 99.19%].

### Step 4—Synthesis of Host 1 (D1)

To a degassed mixture of Intermediate 5 (18 g, 0.05 mol) and dibenzothiophene-4 boronic acid (18.3 g, 0.05 mol) in toluene (360 mL), was added S-phos (0.43 g, 1.10 mmol) and Pd₂(dba)₃ (0.41 g, 0.53 mmol) at 60° C. A degassed solution of 25% tetraethyl ammonium hydroxide (124 mL, 0.21 mol) was added and the reaction mixture refluxed at 110° C. for 18 h. The reaction mixture was filtered, washed with toluene and the organic phase washed with water (400 mL), brine (300 mL), dried over sodium sulphate and concentrated. The crude product was purified by silica column chromatography (5% EtOAc in hexane), recrystallized from hot toluene/acetonitrile and finally dissolved in toluene, washed with concentrated sulfuric acid, and concentrated to give 15.5 g of Host 1 (D1) [HPLC: 99.91%].

### Host 2 (D11)

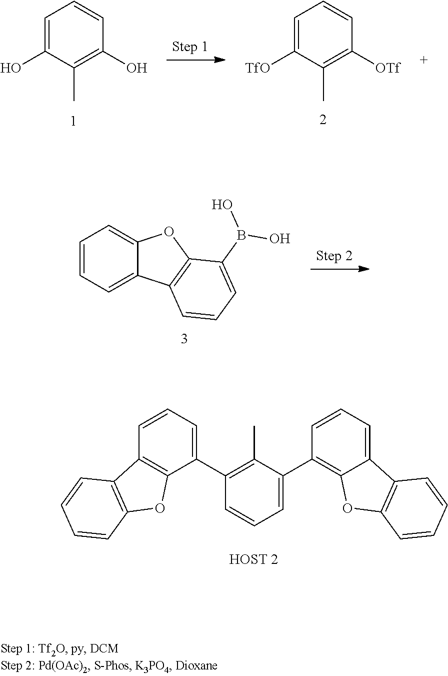

### Step 1—Synthesis of Intermediate 2

To a solution of methyl-resorcinol and pyridine in DCM at 0° C., triflic anhydride (23.9 g, 0.08 mmol) was added dropwise, maintaining a temperature <10° C. After warming to room temperature and stirring for a further 22 h, the reaction mixture was filtered through silica, the combined eluants were concentrated to yield an orange oil which crystallized upon standing to give 15.2 g of intermediate 2 [GCMS: m/z=388; ¹H NMR (600 MHz, CDCl3): δ 7.4-7.33 (m, 3H), 2.39 (s, 3H)].

### Step 2—Synthesis of Host 2 (D11)

To a degassed solution of Intermediate 2 (10.0 g, 25.76 mmol), dibenzofuran-2-boronic acid (13.65 g, 64.39 mmol) and potassium phosphate tribasic (16.40 g, 77.27 mmol) in dioxane (400 mL) was added Pd(OAc)₂ (116 mg, 0.52 mmol) and S-phos (211 mg, 0.52 mmol) and the reaction mixture heated under reflux for 4 days. After cooling to room temperature, the mixture was filtered through celite, and purified by silica column chromatography (heptane/toluene), followed by recrystallization from heptane/toluene and vacuum sublimation (225° C.) to yield 3.7 g of Host 2 (D11) [mpt: 170° C.; HPLC: 99.74%; LCMS: m/z=424 [M⁺]; ¹H NMR (600 MHz, CDCl3): δ 8.025 (d, J=8.0 Hz, 2H), 8.005 (d, J=8.0 Hz, 2H), 7.610 (d, J=8.5 Hz, 2H), 7.56-7.45 (m, 9H), 7.380 (t, J=8.5 Hz, 2H), 2.126 (s, 3H)].

### Host 3 (D8)

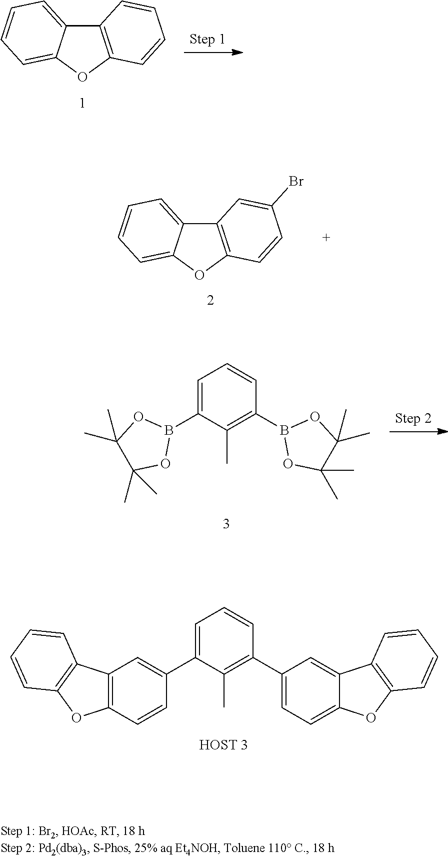

### Step 1—Synthesis of Intermediate 2

Bromine (6.1 mL, 0.12 mol) was added dropwise to a mixture of dibenzofuran (20 g, 0.12 mol) in acetic acid (200 mL) at room temperature. After stirring for 18 h, the reaction mixture was filtered, washed with water (100 mL) and dried. The resulting solid was dissolved in EtOAc (200 ml), washed with sodium thiosulphate solution (10 g in 200 mL of water), water (200 L), dried over sodium sulphate and concentrated. The crude product was purified by hot toluene followed by hot hexane recrystallization to yield 11 g of Intermediate 2 [HPLC: 100%; ¹H-NMR (400 MHz, CDCl₃): δ 7.36-7.41 (m, 1H), 7.46-7.54 (m, 2H), 7.56-7.61 (m, 2H), 7.94 (d, J=7.64 Hz, 1H), 8.10 (s, 1H).

### Step 2—Synthesis of Host 3 (D8)

To a degassed mixture of Intermediate 3 (5 g, 0.02 mol) and Intermediate 2 (15.3 g, 0.045 mol) in toluene (200 mL), was added S-phos (0.16 g, 0.40 mmol) and Pd₂(dba)₃ (0.18 g, 0.20 mmol) at 60° C. A degassed solution of 25% tetraethyl ammonium hydroxide (47.6 mL, 0.08 mol) was added and the reaction mixture refluxed at 110° C. for 16 h. The crude product was filtered through a Florosil-silica plug and purified by silica column chromatography (25% CHCl₃ in hexane), recrystallized from hot toluene/acetonitrile followed and finally filtered from hot DCM and concentrated to give 4.2 g of Host 3 (D8) [HPLC: 99.8%; ¹H-NMR (400 MHz, CDCl₃): δ 2.20 (s, 3H), 7.36-7.40 (m, 5H), 7.48-7.52 (m, 4H), 7.61-7.66 (m, 4H), 7.98-7.99 (m, 4H)].

Synthesis of Blue Phosphorescent Emitter 1 is disclosed in WO2016046572.

Blue Phosphorescent Emitter 2 was prepared in the same way, but using a triazole intermediate as shown below:

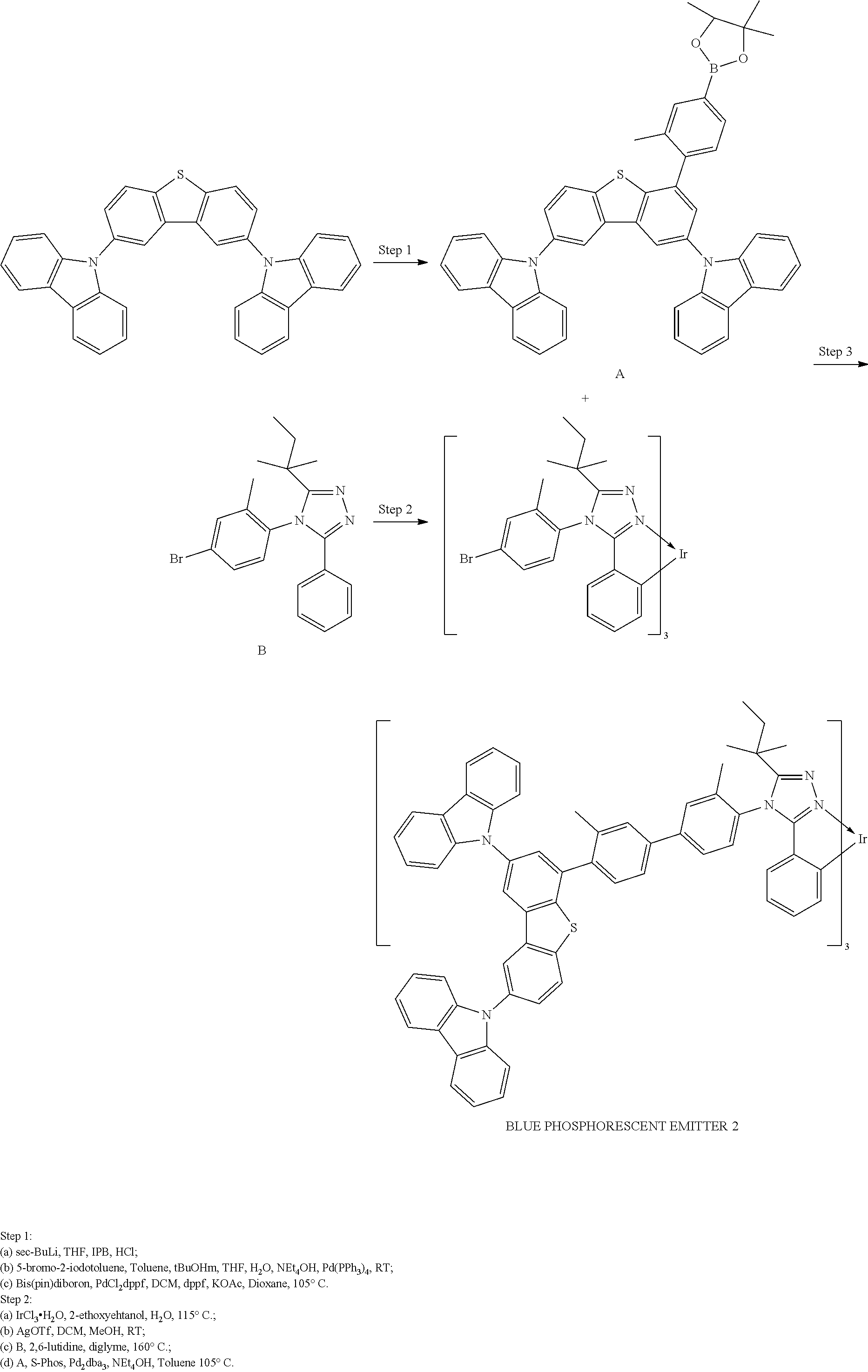

Tg Measurement

Tg values given herein were measured by differential scanning calorimetry using a PerkinElmer DSC8500 according to the method described below.

The apparatus was purged with nitrogen gas at 20 ml/min, the host (5 to 10 mg) was placed in a sample pan and loaded into the sample furnace and a reference pan (containing no sample) was loaded into in the reference furnace.

Heating and cooling was conducted according to the following temperature program:
1) Hold for 1 min at −50° C. and switch the gas to Helium at 20 mL/min
2) Heat from −50° C. to 300° C. at 300° C./min and hold for 1 min at 300° C.
3) Cool from 300° C. to −50° C. at 300° C./min and hold for 1 min at −50° C.
4) Heat from −50° C. to 300° C. at 20° C./min and hold for 1 min at 300° C.
5) Cool from 300° C. to −50° C. at 20° C./min and hold for 1 min at −50° C.
6) Heat from −50° C. to 300° C. at 100° C./min and hold for 1 min at 300° C.
7) Cool from 300° C. to −50° C. at 100° C./min
8) Hold for 1 min at −50° C. and switch the gas to Nitrogen at 20 ml/min.

The Tg of the host was determined from the rising temperature ramp of 20° C./min, and the falling temperature ramp of 100° C./min was used to confirm the Tg event.

### Device Example 1

A substrate carrying ITO (45 nm) was cleaned using UV/Ozone. A hole injection layer was formed to a thickness of about 35 nm by spin-coating a formulation of a hole-injection material available from Nissan Chemical Industries. A red light-emitting layer was formed to a thickness of about 20 nm by spin-coating a red-emitting hole-transporting polymer comprising fluorene repeat units, amine repeat units of formula (VII) and Red Phosphorescent Repeat Unit 1 and substituted with crosslinkable groups, and crosslinking the polymer by heating at 180° C. A green and blue light-emitting layer was formed to a thickness of about 70 nm by spin-coating Host 1 (74 wt %), Green Phosphorescent Emitter 1 (1 wt %) and Blue Phosphorescent Emitter 1 (25 wt %). A layer of compound HB1 was evaporated onto the light-emitting layer. An electron-transporting layer was formed by spin-coating a polymer comprising Electron-Transporting Repeat Unit 1 onto the layer of compound HB1 from a 2,2,3,3,4,4,5,5-octafluoro-1-pentanol solution. This partially formed device was heated to 130-150° C. on a hotplate. A cathode was formed by evaporating a layer of sodium fluoride of about 2 nm thickness, a layer of aluminium of about 100 nm thickness and a layer of silver of about 100 nm thickness.

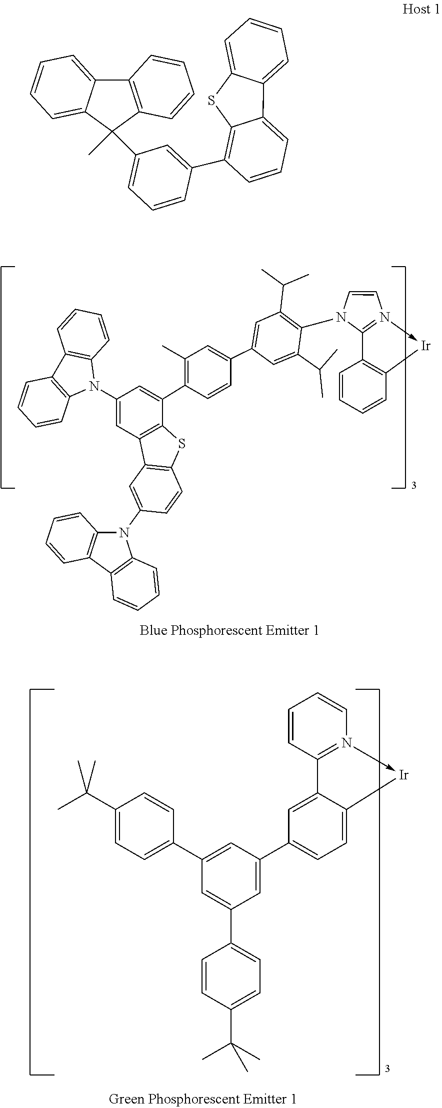

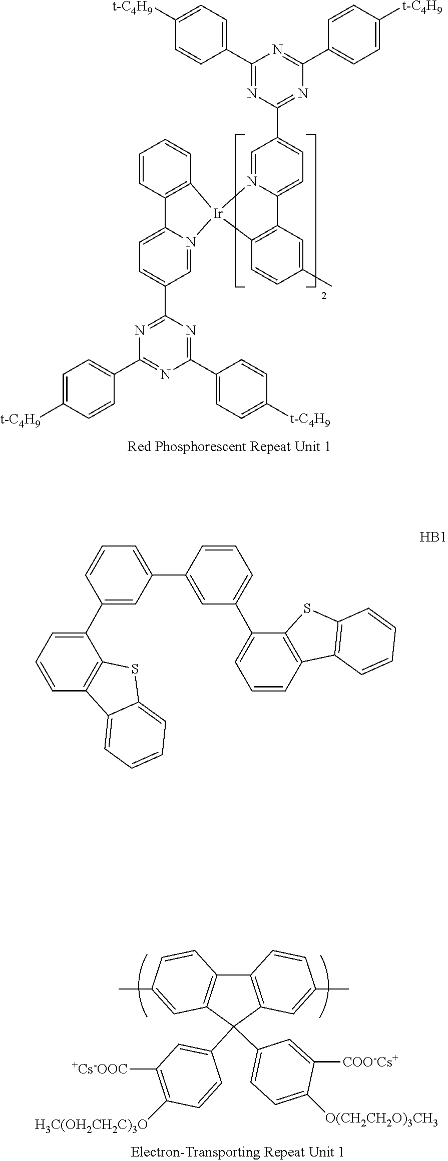

Comparative Device 1

For the purpose of comparison, a device was formed as described for Device Example 1 except Blue Phosphorescent Emitter 1 was replaced with Comparative Blue Emitter 1:

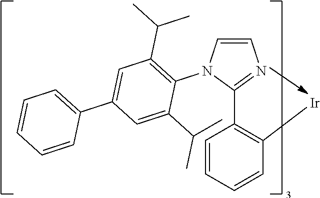

Comparative Blue Emitter 1

With reference to FIG. 2, the light emitted from Device Example 1 has a much stronger blue and green component than Comparative Device 1.

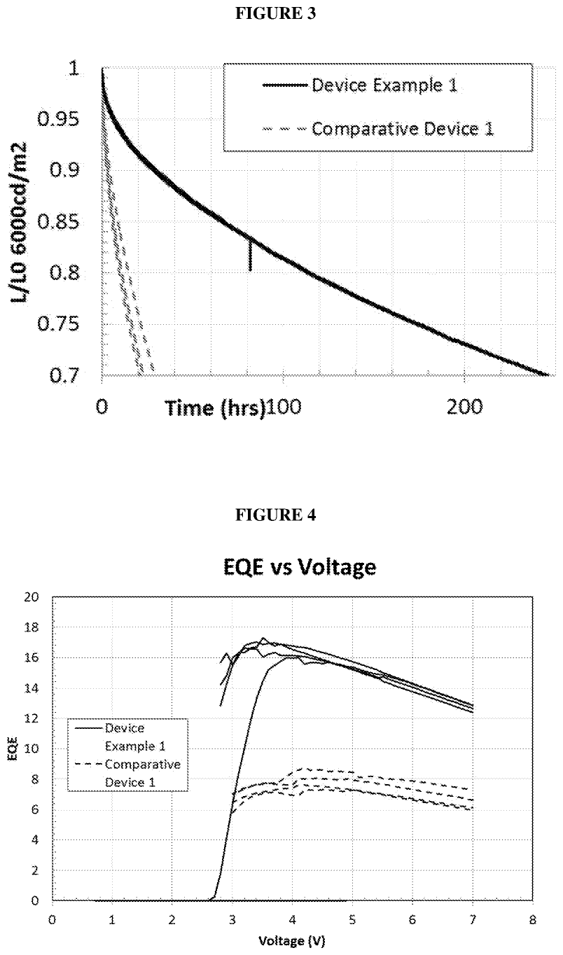

With reference to FIG. 3, the time taken for brightness to fall to 70% of a starting value at constant current is much longer for Device Example 1 than for Comparative Device 1.

With reference to FIG. 4, external quantum efficiency of Device Example 1 is considerably higher than that of Comparative Device 1.

### Device Example 2

A device was prepared as described for Device Example 1 except that the layer of compound HB1 was not included.

Comparative Device 2

A device was prepared as for Device Example 2 except that Comparative Host 1 was used in place of Host 1:

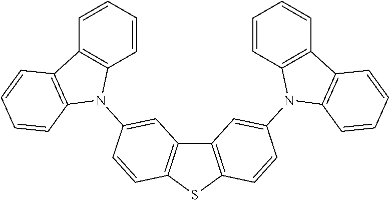 [SMILES: c1ccc2c(c1)c1ccccc1n2-c1ccc2sc3ccc(-n4c5ccccc5c5ccccc54)cc3c2c1]

Comparative Host 1

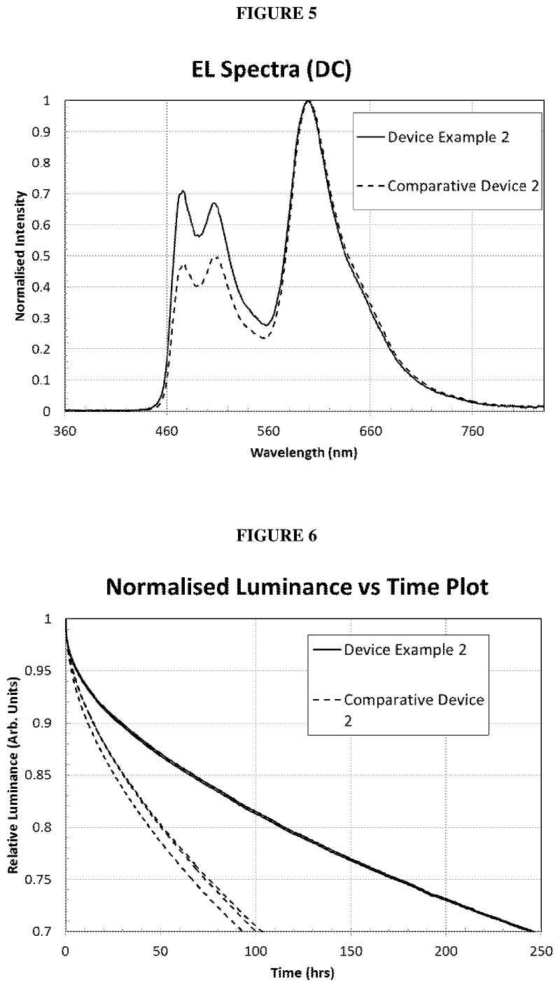

With reference to FIG. 6 the time taken for brightness to fall to 70% of a starting value at constant current is much longer for Device Example 2 as compared to Comparative Device 2, even though the proportion of shorter wavelength (higher energy) luminance is greater for Device Example 2 than Comparative Device 2 as shown in FIG. 5.

### Example 3

The stabilities of compositions of Host 1, 2 and 3 and phosphorescent emitters were measured by irradiating the compositions with ultraviolet light and measuring the time taken for luminance of the composition to fall to 70% of an initial value.

Films of 80 nm thickness were spun on glass substrates and encapsulated, with the inclusion of a getter. The films were irradiated using a laser diode of wavelength 405 nm, focused to a spot size of 1 mm². The total photoluminescence counts were integrated over the range 450-650 nm using a confocal geometry and an ocean optics USB200 spectrometer. The time taken for the total PL counts to fall to 70% of the initial value (T70) was recorded.

The intensity of irradiation was adjusted so that the luminance of the film comprising the comparative compound reached T70 over a timescale of 1 to 2 hrs. The film comprising the example compound was then irradiated in the same manner, with the intensity of the 405 nm radiation adjusted so as to give the same initial number of photoluminescence counts as that of the film comprising the comparative compound between 450 and 650 nm

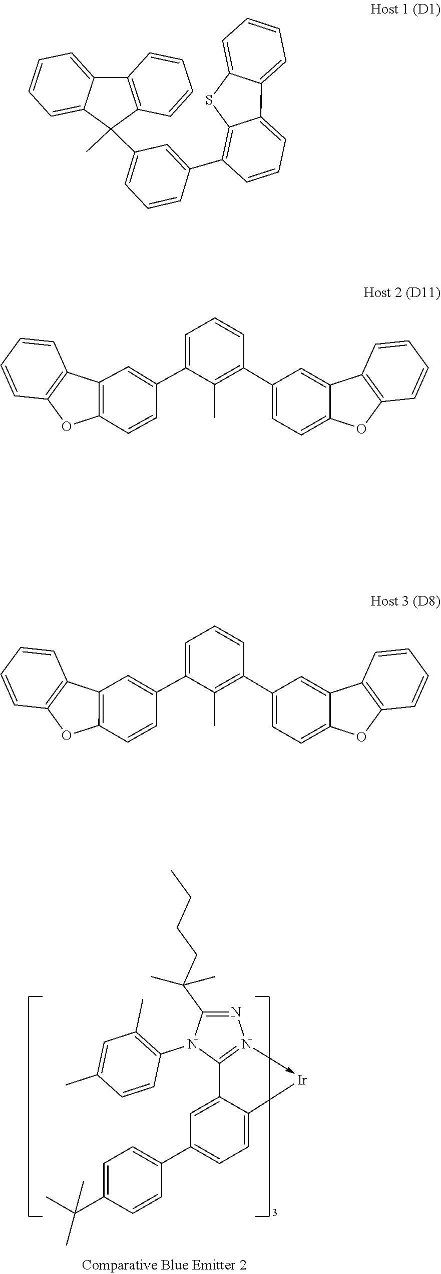

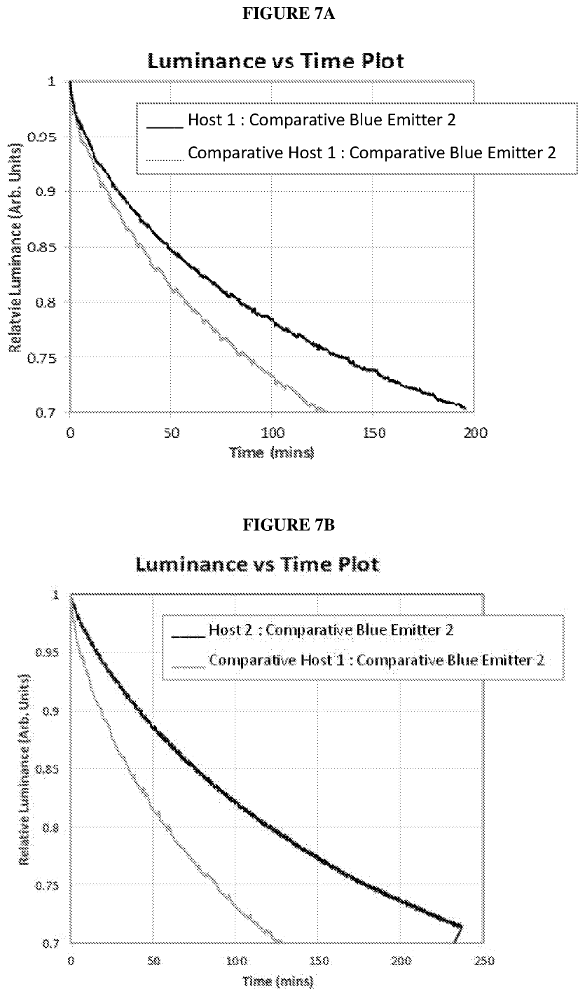

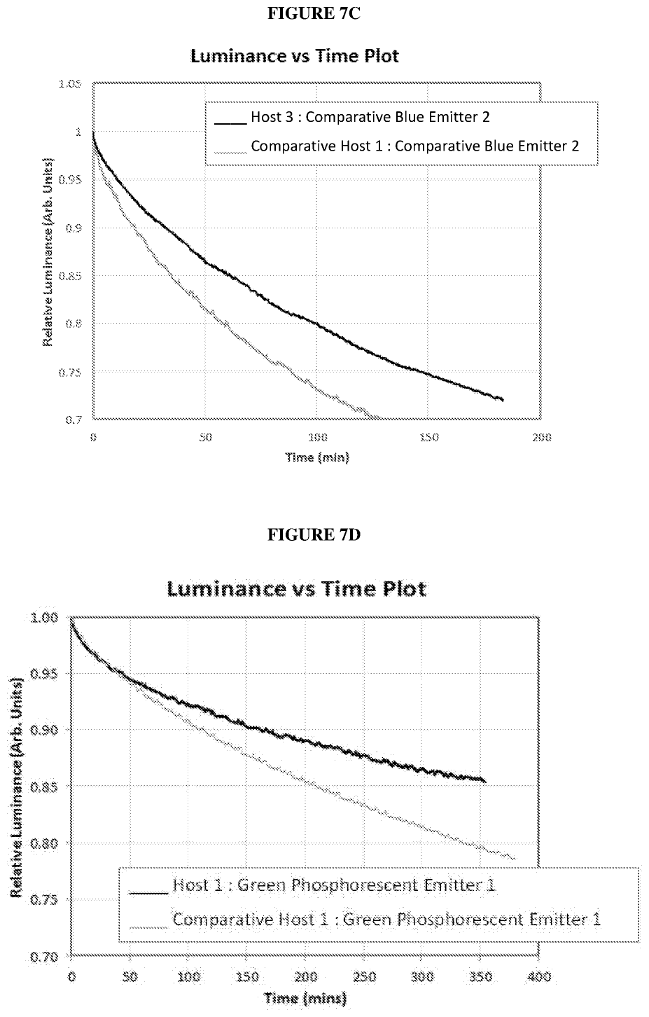

With reference to FIGS. 7A, 7B and 7C, low Tg Host 1, Host 2 and Host 3 show improved photostability for EML films when doped with Comparative Blue Emitter 2 as compared to Comparative Host 1.

With reference to FIG. 7D, the low Tg Host 1 shows improved photostability when doped with Comparative Green Emitter 1 as compared to Comparative Host 1.

### Example 4

UV stability was measured as described in Example 3, except that Comparative Blue Emitter 2 was replaced by Blue Phosphorescent Emitter 1.

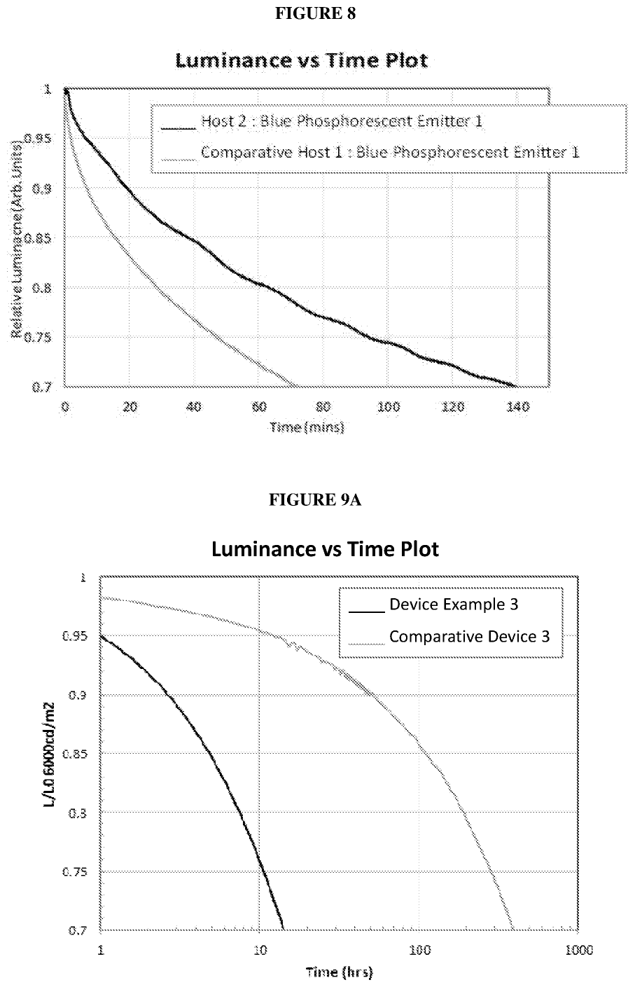

With reference to FIG. 8, the low Tg Host 2 shows improved photostability for EML films as compared to Comparative Host 1, when doped with Blue Phosphorescent Emitter 1.

### Device Example 3

A device was prepared as described for Device Example 1 except that the Blue Phosphorescent Emitter 1 was excluded and Host 2 was used in place of Host 1.

Comparative Device 3

A device was prepared as for Device Example 3 except that Comparative Host 1 was used in place of Host 2.

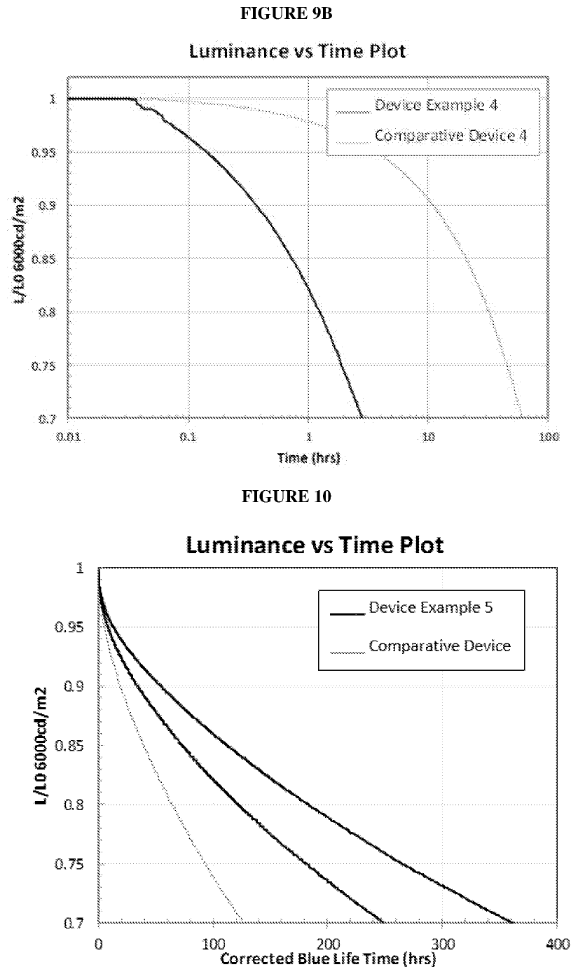

With reference to FIG. 9A, the time taken for brightness to fall to 70% of a starting value at a constant current is much longer for Comparative Host 1 than for Host 2 when doped with Green Phosphorescent Emitter 1.

### Device Example 4

A device was prepared as described for Device Example 1 except that Host 2 replaced Host 1, Blue Phosphorescent Emitter 1 was replaced with Comparative Blue Emitter 3, and Green Phosphorescent Emitter 1 was excluded.

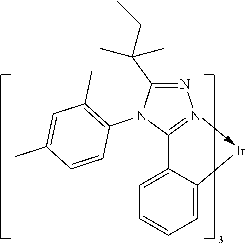

Comparative Blue Emitter 3

Comparative Device 4

A device was prepared as for Device Example 4 except that Comparative Host 1 was used in place of Host 2.

With reference to FIG. 9B, the time taken for brightness to fall to 70% of a starting value at a constant current is much longer for Comparative Host 1 than for Host 2 when doped with Comparative Blue Emitter 3.

### Device Example 5

A device was prepared as described for Device Example 1 except that Host 1 was replaced by either Host 2 or Host 3 and Green Phosphorescent Emitter 1 was excluded.

Comparative Device 5

A device was prepared as for Device Example 5 except that Comparative Host 1 was used in place of Host 2 or 3.

With reference to FIG. 10, the time taken for brightness to fall to 70% of a starting value at a constant current was longer for Host 2 and Host 3 than for Comparative Host 1 when doped with Blue Phosphorescent Emitter 1.

A correction was made to the time taken for brightness to fall to 70% to compensate for the relative intensities of the blue wherein the fraction of blue emission in the emission spectrum having a wavelength in the range of 400-490 nm of a test device is compared to that of a reference spectrum at the desired colour point. If the spectrum of the test device has more blue emission than the reference, the correction increases the lifetime and if input has less blue emission the correction reduces the lifetime. This is done as luminance is a function of the eye response, a blue device with increased green emission (higher CIEy) will require a lower photon output than a bluer device to achieve the same luminance. This means that when driven from the same initial luminance, a blue device with a higher CIEy is required to produce fewer photons and hence takes longer to degrade.

### Device Example 6

A device was prepared as described for Device Example 5 except that Blue Phosphorescent Emitter 1 was replaced by Blue Phosphorescent Emitter 2.

Comparative Device 6

A device was prepared as for Device Example 6 except that Comparative Host 1 was used in place of Host 2 or 3.

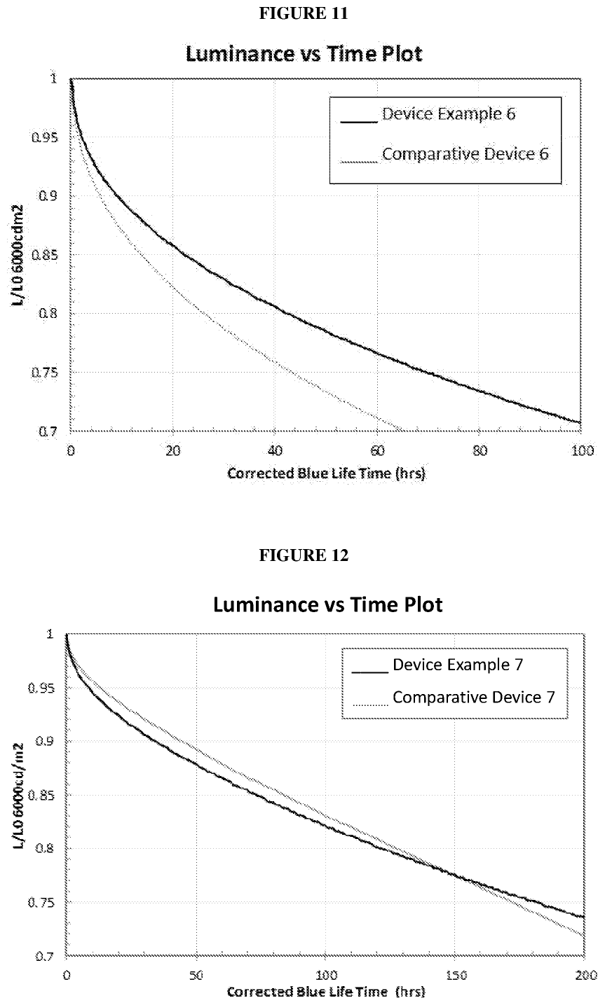

With reference to FIG. 11, the time taken for brightness to fall to 70% of a starting value at a constant current is longer for Host 2 than for Comparative Host 1 when doped with Blue Phosphorescent Emitter 2. The life time value is corrected as described in Device Example 5.

### Device Example 7

A device was prepared as described for Device Example 5 except that Host 2 was used with Blue Phosphorescent Emitter 1.

Comparative Device 7

A device was prepared as described for Device Example 6 except that Blue Phosphorescent Emitter 2 was replaced by Blue Phosphorescent Emitter 3.

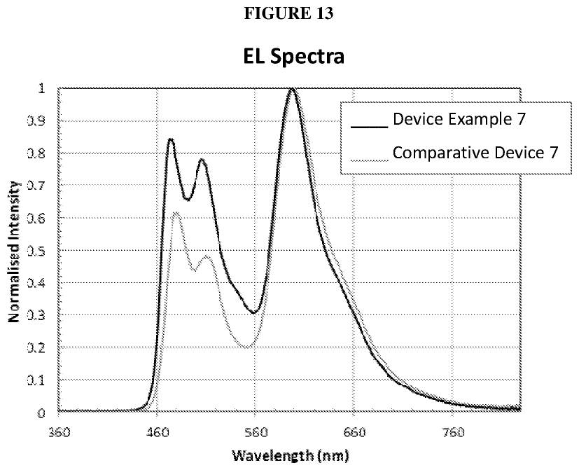

With reference to FIG. 12 the time taken for brightness to fall to 70% of a starting constant current for Device Example 5 is comparable to Comparative Device 7 even though the proportion of shorter wavelength (higher energy) luminance is greater for Device Example 7 than Comparative Device 7 as shown in FIG. 13. The life time value is corrected as described in Device Example 5.

1. A composition comprising a semiconducting host compound having a glass transition temperature (Tg) of less than 100° C., and a phosphorescent compound wherein the phosphorescent compound is a metal complex of formula (II):
M(L¹)p(L²)q (II)
wherein
M is Ir (III) or Pt (II);
L¹ is a bidentate ligand of formula (II): 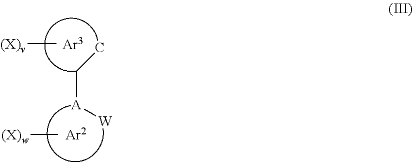
wherein:
Ar² is a 5-20 membered heteroaryl group;
Ar³ is a C₆₋₂₀ aryl group or a 5-20 membered heteroaryl group;
A is C or N;
W is N if A is C and W is a carbene C atom if A is N;
L² is a bidentate ligand which is different from L¹;
p is at least 1;
q is 0, 1 or 2;
each X independently comprises an aromatic or heteroaromatic group Ar⁵ which is unsubstituted or substituted with one or more substituents;
v and w are each independently 0 or 1 with the proviso that at least one of v and w is 1;
the sum of the number of rings comprised in the one or more X groups of formula (II) is at least 12; and
at least 75% of the mass of each X is made up of the mass of the aromatic or heteroaromatic ring atoms of Ar⁵.

2. The composition according to claim 1, wherein the semiconducting host compound has formula (I):
Ar²⁰—(Ar¹⁰)ᵤ—Ar³⁰ (I)
wherein
Ar¹⁰ independently in each occurrence is an arylene which is unsubstituted or substituted with one or more substituents;
Ar²⁰ is a group of formula (Ia): 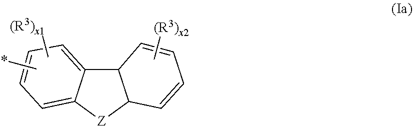
wherein
Z is O or S;
R³ is a substituent
x₁ is 0, 1, 2 or 3;
x₂ is 0, 1, 2, 3 or 4;
u is 1, 2, or 3;
Ar³⁰ is a group of formula (Ib) or (Ic): 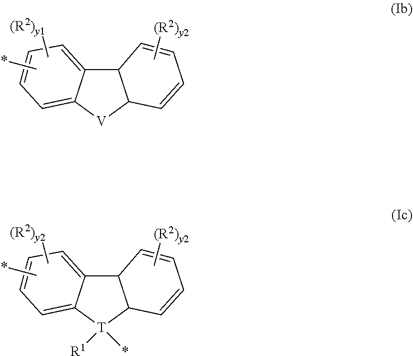
wherein
V is O, S, —C(R⁹)₂— or —Si(R¹¹)₂—;
T is C or Si;
R¹, R², R⁹ and R¹¹ are substituents;
y₁ is 0, 1, 2 or 3; and
y₂ is 1, 2, 3 or 4.

3. A composition according to claim 2, wherein u is 1.

4. A composition according to claim 2 wherein Ar¹⁰ is phenylene.

5. A composition according to claim 2 wherein u is 1 and Ar¹⁰ is 1,3-phenylene.

6. A composition according to claim 2 wherein u is 3, and (Ar¹⁰)ᵤ has formula (Xm) or (Xn): 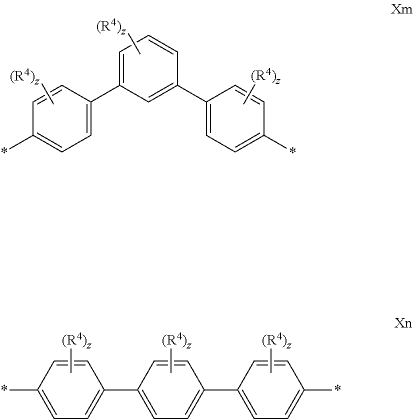
wherein R⁴ in each occurrence is independently a substituent and z in each occurrence is independently 0, 1, 2, 3 or 4.

7. A composition according to claim 1 wherein the semiconducting host compound is not a polymer.

8. A composition according to claim 2, wherein the compound of formula (I) has formula (Id):

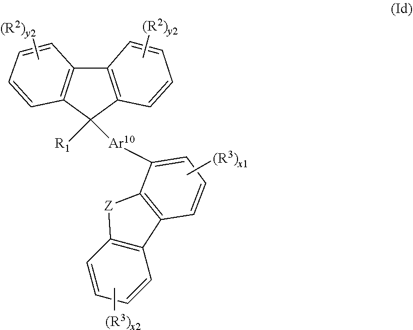

9. (canceled)

10. A composition according to claim 1 wherein x=0 or y=0.

11. A composition comprising according to claim 2, wherein the compound of formula (I) is a compound of formula (Ie) or (If):

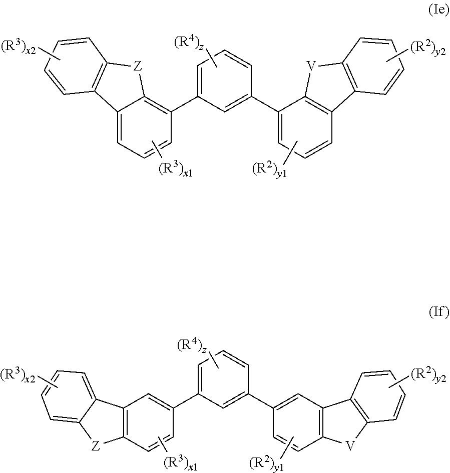

12. A composition according to claim 1 wherein W is N, A is C and Ar² is a 5- or 6-membered heteroaromatic group having ring atoms selected from C and N.

13. A composition according to claim 1 wherein Ar³ is phenyl.

14. A composition according to claim 1 wherein X is a group of formula (V):
—(Ar⁵)ₘ, (V)
wherein Ar⁵ in each occurrence is independently an aromatic or heteroaromatic group which is unsubstituted or substituted with one or more substituents, and m is at least 3.

15. A composition according to claim 14 wherein the group of formula (V) is a branched chain of Ar⁵ groups.

16. A composition according to claim 14 or 15 wherein each Ar⁵ group is selected from the group consisting of benzene, dibenzothiophene, dibenzofuran and carbazole, each of which is independently unsubstituted or substituted with one or more substituents.

17. A composition according to claim 1 wherein the compound of formula (II) is a blue phosphorescent compound.

18. A formulation comprising a composition according to claim 1 and one or more solvents.

19. An organic light-emitting device comprising an anode, a cathode and a light-emitting layer between the anode and the cathode wherein the light-emitting layer comprises a composition according to claim 1.

20. An organic light-emitting device according to claim 19 wherein the light-emitting layer is directly adjacent to a hole-transporting layer disposed between the anode and the light-emitting layer.

21. (canceled)

22. A composition comprising a compound of formula (IV) and a phosphorescent compound of formula (II): 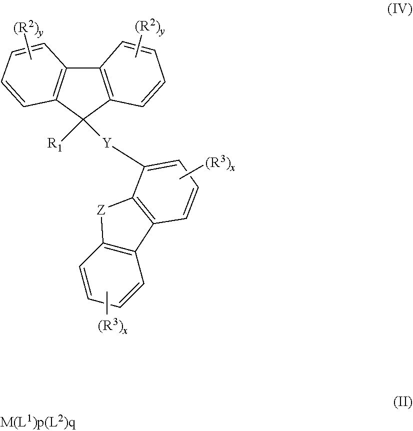
wherein:
Z is O or S;
R¹, R² and R³ are each independently a substituent;
Y is a direct bond or an arylene or heteroarylene group;
x is 0, 1, 2, 3 or 4;
y is 0, 1, 2, 3 or 4;
M is Ir (III) or Pt (II);
L¹ is a bidentate ligand of formula (III): 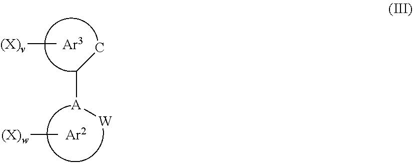
wherein:
Ar² is a 5-20 membered heteroaryl group;
Ar³ is a C₆₋₂₀ aryl group or a 5-20 membered heteroaryl group;
A is C or N;
W is N if A is C and W is a carbene C atom if A is N;
L² is a bidentate ligand which is different from L¹;
p is at least 1;
q is 0, 1 or 2;
each X independently comprises an aromatic or heteroaromatic group Ar⁵ which is unsubstituted or substituted with one or more substituents;
v and w are each independently 0 or 1 with the proviso that at least one of v and w is 1;
the sum of the number of rings comprised in the one or more X groups of formula (II) is at least 12; and
at least 75% of the mass of each X is made up of the mass of the aromatic or heteroaromatic ring atoms of Ar⁵.
